(19) United States
(12) Patent Application Publication
(10) Pub. No.: US 2013/0261433 A1
(43) Pub. Date: Oct. 3, 2013

(54) HAPTIC SIMULATION AND SURGICAL LOCATION MONITORING SYSTEM AND METHOD

(71) Applicant: NAVIDENT TECHNOLOGIES, INC., North Vancouver (CA)

(72) Inventor: Ehud Daon, North Vancouver (CA)

(73) Assignee: Navident Technologies, Inc., North Vancouver (CA)

(21) Appl. No.: 13/735,487

(22) Filed: Jan. 7, 2013

Related U.S. Application Data
(60) Provisional application No. 61/616,704, filed on Mar. 28, 2012.

(51) Int. Cl. A61B 19/00

(52) U.S. Cl. CPC A61B 19/00
USPC 600/424

(57) ABSTRACT

A system and method for monitoring a surgical site employs a fiducial reference with attached tracking marker. The marker may be tracked by a tracker. Image information about the surgical site is provided to a controller that updates a model of the surgical site derived from a prior scan of the surgical site. The system and method allow the user to select between monitoring the real surgery and performing virtual surgery using a digital manipulator device. The system may be operated with the user located remotely from the surgical site and operating a remotely operable surgical instrument controlled by the digital manipulator device. The system can select between employing a stationary model and a model that is updated in real time based on the real time image information from the surgical site. The manipulator device may be a haptic feedback device and the haptic feedback may be optionally employed.

### CROSS REFERENCE TO RELATED APPLICATIONS

The present application claims priority under 35 U.S.C. §119(e) of U.S. Provisional Patent Application Ser. No. 61/616,704, filed on Mar. 28, 2012 and titled “Haptic Simulation and Surgical Location Monitoring System” the disclosure of which is expressly incorporated by reference herein.

### BACKGROUND OF THE INVENTION

1. Field of the Invention

The invention relates to location monitoring hardware and software systems. More specifically, the field of the invention is that of surgical equipment and software for monitoring surgical conditions.

2. Description of the Related Art

Visual and other sensory systems are known, with such systems being capable of both observing and monitoring surgical procedures. With such observation and monitoring systems, computer aided surgeries are now possible, and in fact are being routinely performed. In such procedures, the computer software interacts with both clinical images of the patient and observed surgical images from the current surgical procedure to provide guidance to the physician in conducting the surgery. In addition, haptic feedback systems are known, and are used daily. These systems provide force feedback based on three-dimensional (3D) data that resides in a computer. With this relatively new computer implemented technology, further improvements may further advance the effectiveness of surgical procedures.

### SUMMARY OF THE INVENTION

The present invention is a surgical hardware and software monitoring system and method that allows for surgical planning. The present invention further allows for simulation of real cases, and then proceed to perform the same surgery on a real patient using the simulated data. In one embodiment, the patient data may be used as a basis for plannning a surgical procedure. A 3D virtual model may be used to simulate the contemplated surgical procedures and warn the physician with haptic feedback regarding possible boundary violations that would indicate an inappropriate location in a surgical procedure. In another embodiment, the hardware may track the movement of instruments during the procedure and in reference to the model to enhance observation of the procedure. In this way, physicians are provided an additional tool to improve surgical planning and performance by rehearsing difficult procedures and then having a monitoring system to monitor performance of the same live surgery.

The system uses a particularly configured fiducial reference, to orient the monitoring system with regard to the critical area. The fiducial reference is attached to a location near the intended surgical area. For example, in the example of a dental surgery, a splint may be used to securely locate the fiducial reference near the surgical area. The fiducial reference may then be used as a point of reference, or a fiducial, for the further image processing of the surgical site. The fiducial reference may be identified relative to other portions of the surgical area by having a recognizable fiducial marker apparent in the scan.

Embodiments of the invention involve systems for automatically computing the three-dimensional location of the patient using a tracking device that may include a tracking marker. The tracking marker may be attached in fixed spatial relation either directly to the fiducial reference, or attached to the fiducial reference via a tracking pole that itself may have a distinct three-dimensional shape. In the dental surgery example, a tracking pole is mechanically connected to the base of the fiducial reference that is in turn fixed in the patient's mouth. Each tracking pole device has a particular observation pattern, located either on itself or on a suitable tracking marker, and a particular geometrical connection to the base, which the computer software recognizes as corresponding to a particular geometry for subsequent location calculations. Although individual tracking pole devices have distinct configurations, they may all share a common connection base configuration and thus may be used with any keyucial reference. The particular tracking information calculations are dictated by the particular tracking pole used, and actual patient location is calculated accordingly. Thus, tracking pole devices may be interchanged and calculation of the location remains the same. This provides, in the case of dental surgery, automatic recognition of the patient head location in space. Alternatively, a sensor device, or a tracker, may be in a known position relative to the fiducial key and its tracking pole, so that the current data image may be mapped to the scan image items.

The fiducial reference and each tracking pole or associated tracking marker may have a pattern made of radio opaque material so that when imaging information is scanned by the software, the particular items are recognized. Typically, each instrument used in the procedure has a unique pattern on its associated tracking marker so that the tracker information identifies the instrument. The software creates a model of the surgical site, in one embodiment a coordinate system, according to the location and orientation of the patterns on the fiducial reference and/or tracking pole(s) or their attached tracking markers. By way of example, in the embodiment where the fiducial reference has an associated pre-assigned pattern, analysis software interpreting image information from the tracker may recognize the pattern and may select the site of the base of the fiducial to be at the location where the fiducial reference is attached to a splint. If the fiducial key does not have an associated pattern, a fiducial site is designated. In the dental example this may be at a particular spatial relation to the tooth, and a splint location can be automatically designed for placement of the fiducial reference.

One aspect of the invention provides a surgical monitoring system comprising a fiducial reference configured for removably attaching to a location proximate a surgical site, for having a three-dimensional location and orientation determinable based on scan data of the surgical site, and for having the three-dimensional location and orientation determinable based on image information about the surgical site; a tracker arranged for obtaining the image information; and a controller configured for spatially relating the image information to the scan data and for determining the three-dimensional location and orientation of the fiducial reference. In one embodiment of the invention the fiducial reference may be rigidly and removably attachable to a part of the surgical site. In such an embodiment the fiducial reference may be repeatably attachable in the same three-dimensional orientation to the same location on the particular part of the surgical site.

The fiducial reference involves at least one of marked and shaped for having at least one of its location and its orientation determined from the scan data and to allow it to be uniquely identified from the scan data. The surgical monitoring system further comprises a first tracking marker in fixed three-dimensional spatial relationship with the fiducial reference, wherein the first tracking marker is configured for having at least one of its location and its orientation determined by the controller based on the image information and the scan data. The first tracking marker may be configured to be removably and rigidly connected to the fiducial reference by a first tracking pole. The first tracking pole can have a three-dimensional structure uniquely identifiable by the controller from the image information. The three-dimensional structure of the first tracking pole allows its three-dimensional orientation of the first tracking pole to be determined by the controller from the image information.

The first tracking pole and fiducial reference may be configured to allow the first tracking pole to connect to a single unique location on the fiducial reference in a first single unique three-dimensional orientation. The fiducial reference may be configured for the attachment in a single second unique three-dimensional orientation of at least a second tracking pole attached to a second tracking marker. The first tracking marker may have a three-dimensional shape that is uniquely identifiable by the controller from the image information. The first tracking marker can have a three-dimensional shape that allows its three-dimensional orientation to be determined by the controller from the image information. The first tracking marker may have a marking that is uniquely identifiable by the controller and the marking may be configured for allowing at least one of its location and its orientation to be determined by the controller based on the image information and the scan data.

The fiducial reference may be a multi-element fiducial pattern comprising a plurality of pattern segments and every segment is individually configured for having a segmental three-dimensional location and orientation determinable based on scan data of the surgical site, and for having the segmental three-dimensional location and orientation determinable based on image information about the surgical site. The plurality of pattern segments may have unique differentiable shapes that allow the controller to identify them uniquely from at least one of the scan data and the image information. Tracking markers may be attached to at least a selection of the pattern segments, the tracking markers having at least one of identifying marks and orientation marks that allow their three-dimensional orientations to be determined by the controller from the image information. The controller may be configured for determining the locations and orientations of at least a selection of the pattern segments based on the image information and the scan data. The controller may be configured for calculating of the locations of anatomical features in the proximity of the multi-element fiducial pattern.

The surgical monitoring system may comprise further tracking markers attached to implements proximate the surgery site and the controller may be configured for determining locations and orientations of the implements based on the image information and information about the further tracking markers.

Another aspect of the invention provides a method for relating in real time the three-dimensional location and orientation of a surgical site on a patient to the location and orientation of the surgical site in a scan of the surgical site. The method comprises removably attaching a fiducial reference to a fiducial location on the patient proximate the surgical site. The scan is performed with the fiducial reference attached to the fiducial location to obtain scan data. Three-dimensional location and orientation of the fiducial reference is determined from the scan data. Real time image information of the surgical site is obtained and the three-dimensional location and orientation of the fiducial reference is determined in real time from the image information. A spatial transformation matrix is derived for expressing in real time the three-dimensional location and orientation of the fiducial reference as determined from the image information in terms of the three-dimensional location and orientation of the fiducial reference as determined from the scan data.

Obtaining real time image information of the surgical site may comprise rigidly and removably attaching to the fiducial reference a first tracking marker in a fixed three-dimensional spatial relationship with the fiducial reference. The first tracking marker may be configured for having its location and its orientation determined based on the image information. Attaching the first tracking marker to the fiducial reference may comprise rigidly and removably attaching the first tracking marker to the fiducial reference using a tracking pole. Obtaining real time image information of the surgical site may comprise rigidly and removably attaching to the fiducial reference a tracking pole in a fixed three-dimensional spatial relationship with the fiducial reference. Further, the tracking pole may have a distinctly identifiable three-dimensional shape that allows its location and orientation to be uniquely determined from the image information. In the case where the fiducial reference is a multi-element fiducial pattern comprising a plurality of pattern segments individually locatable based on the scan data, determining the three-dimensional location and orientation of the fiducial reference from the scan data may comprise determining the three-dimensional location and orientation of at least a selection of the plurality of pattern segments from the scan data. Determining in real time the three-dimensional location and orientation of the fiducial reference from the image information may comprise determining the three-dimensional location and orientation of the at least a selection of the plurality of pattern segments from the image information.

Another aspect of the invention provides a method for tracking in real time changes in a surgical site. Such a method comprises removably attaching a multi-element fiducial reference to a fiducial location on the patient proximate the surgical site, the multi-element fiducial reference comprising a plurality of pattern segments individually locatable based on scan data. A scan is performed with the fiducial reference attached to the fiducial location to obtain the scan data. Three-dimensional locations and orientations of at least a selection of the pattern segments are determined from the scan data. Real time image information is obtained from the surgical site. Three-dimensional locations and orientations of the at least a selection of the pattern segments is determined in real time from the image information. The spatial distortion of the surgical site is derived in real time by comparing in real time the three-dimensional locations and orientations of the at least a selection of the pattern segments as determined from the image information with the three-dimensional locations and orientations of the at least a selection of the pattern segments as determined from the scan data.

A further aspect of the invention provides a method for real time monitoring the position of an object in relation to a surgical site of a patient. This method comprises removably attaching a fiducial reference to a fiducial location on the patient proximate the surgical site. A scan is performed with the fiducial reference attached to the fiducial location to obtain scan data. Three-dimensional location and orientation of the fiducial reference is determined from the scan data. Real time image information of the surgical site is obtained. Three-dimensional location and orientation of the fiducial reference is determined in real time from the image information. A spatial transformation matrix is derived for expressing in real time the three-dimensional location and orientation of the fiducial reference as determined from the image information in terms of the three-dimensional location and orientation of the fiducial reference as determined from the scan data. Three-dimensional location and orientation of the object is obtained in real time from the image information and relates the three-dimensional location and orientation of the object to the three-dimensional location and orientation of the fiducial reference as determined from the image information. Determining in real time of the three-dimensional location and orientation of the object from the image information may comprise rigidly attaching a tracking marker to the object.

In one alternative embodiment, the tracker itself is attached to the fiducial reference so that the location of an object having a marker may be observed from a known position.

A still further aspect of the invention a system involves monitoring a surgical site, the system comprising a tracker disposed to monitor the surgical site, the tracker having a field of view; a fiducial reference affixed proximate the surgical site and arranged for moving with the surgical site; a first tracking marker rigidly attached to the fiducial reference, the first tracking marker disposed within the field of view; a surgical instrument disposed within the field of view; a digital manipulator device configured for providing its own real time three-dimensional location and orientation information; and a controller configured for (1) creating a model of the surgical site from scan data of the surgical site; (2) optionally updating in real time the model of the surgical site based on real time image information from the tracker about the surgical site; (3) obtaining the real time three-dimensional location and orientation information of the digital manipulator device; and for (4) displaying simultaneously on a display monitor the model of the surgical site and selectably one of a real time virtual representation of the digital manipulator device and a virtual representation of the surgical instrument. The system may further comprise a second tracking marker attached to the surgical instrument for registering the surgical instrument to the surgical site.

The digital manipulator device may bear a third tracking marker disposed within the field of view for registering the digital manipulator device to the surgical site; and the controller may be further configured for obtaining real time three-dimensional location and orientation information of the digital manipulator device from the real time image information. The digital manipulator device may be a haptic device and the controller may be configured for allowing the user to optionally select haptic feedback, the haptic feedback based on the model.

The system may further comprise a remote communications link, and the controller and the digital manipulator device may disposed remote from the surgical site, the surgical instrument may be a remotely operable surgical instrument; and the remote communications link may be configured for (1) transmitting image information from the tracker to the controller, (2) transmitting three-dimensional location and orientation information about the remotely operable surgical instrument to the controller; and for (3) transmitting control information from the controller to the remotely operable surgical instrument. The remotely operable surgical instrument may bear a fourth tracking marker disposed within the field of view for registering the remotely operable surgical instrument to the surgical site; and the controller may be further configured for obtaining three-dimensional location and orientation information of the remotely operable surgical instrument from the image information.

In a further aspect of the invention a method is presented for monitoring a surgery at a surgical site using a surgical instrument, the surgical instrument bearing a first tracking marker disposed within a field of view of a tracker, the tracker disposed proximate the surgical site, the method comprising, (1) affixing a fiducial reference proximate the surgical site, the fiducial reference arranged for moving with the surgical site; (2) obtaining scan data of the surgical site; (3) creating a model of the surgical site from the scan data; (4) rigidly attaching a second tracking marker to the fiducial reference within the field of view; (5) optionally updating in real time the model based on image information about the surgical site from the tracker; (6) registering a digital manipulator device to the model; (7) obtaining the real time three-dimensional location and orientation information of the digital manipulator device; and (8) displaying simultaneously on a display monitor the model of the surgical site and selectably one of a real time virtual representation of the digital manipulator device and a virtual representation of the surgical instrument. The registering the digital manipulator device to the model may be registering to the model a third tracking marker attached to the digital manipulator device, the third tracking marker being disposed within the field of view. The real time three-dimensional location and orientation information of the digital manipulator device may be obtained from the image information. The method may further comprise selectably providing haptic feedback to a user via the digital manipulator device based on the model.

The method may further comprise controlling the position and orientation of the surgical instrument over a remote communications link by manipulation of the digital manipulator device. The surgical instrument may be a remotely operable surgical instrument configured for receiving control instructions from a controller based on position and orientation information from the digital manipulator device. The method may further comprise sending the image information from the tracker to the controller over the remote communications link.

### BRIEF DESCRIPTION OF THE DRAWINGS

The above mentioned and other features and objects of this invention, and the manner of attaining them, will become more apparent and the invention itself will be better understood by reference to the following description of an embodiment of the invention taken in conjunction with the accompanying drawings, wherein:

FIG. 1 is a schematic diagrammatic view of a network system in which embodiments of the present invention may be utilized.

FIG. 2 is a block diagram of a computing system (either a server or client, or both, as appropriate), with optional input devices (e.g., keyboard, mouse, touch screen, etc.) and output devices, hardware, network connections, one or more processors, and memory/storage for data and modules, etc. which may be utilized as controller and display in conjunction with embodiments of the present invention.

FIGS. 3A-J are drawings of hardware components of the surgical monitoring system according to embodiments of the invention.

FIGS. 4A-C is a flow chart diagram illustrating one embodiment of the registering method of the present invention.

FIG. 5 is a drawing of a dental fiducial key with a tracking pole and a dental drill according to one embodiment of the present invention.

FIG. 6 is a drawing of an endoscopic surgical site showing the fiducial key, endoscope, and biopsy needle according to another embodiment of the invention.

FIGS. 7A and 7B are drawings of a multi-element fiducial pattern comprising a plurality of pattern segments in respectively a default condition and a condition in which the body of a patient has moved to change the mutual spatial relation of the pattern segments.

FIGS. 8A-C is a flow chart diagram illustrating one embodiment of the registering method of the present invention as applied to the multi-element fiducial pattern of FIGS. 7A and 7B.

FIG. 9A-D are drawings of embodiments of the surgical monitoring system of the invention.

FIG. 10 is a flow chart diagram illustrating one embodiment of the virtual and real surgery method of the present invention.

FIG. 11 is a drawing of an embodiment of the remote surgical monitoring system of the invention.

FIG. 12 is a a flow chart diagram illustrating one embodiment of the remote real and virtual surgery method of the invention.

Corresponding reference characters indicate corresponding parts throughout the several views. Although the drawings represent embodiments of the present invention, the drawings are not necessarily to scale and certain features may be exaggerated in order to better illustrate and explain the present invention. The flow charts and screen shots are also representative in nature, and actual embodiments of the invention may include further features or steps not shown in the drawings. The exemplification set out herein illustrates an embodiment of the invention, in one form, and such exemplifications are not to be construed as limiting the scope of the invention in any manner.

### DESCRIPTION OF EMBODIMENTS OF THE PRESENT INVENTION

The embodiments disclosed below are not intended to be exhaustive or limit the invention to the precise form disclosed in the following detailed description. Rather, the embodiments are chosen and described so that others skilled in the art may utilize their teachings.

The detailed descriptions that follow are presented in part in terms of algorithms and symbolic representations of operations on data bits within a computer memory representing alphanumeric characters or other information. The hardware components are shown with particular shapes and relative orientations and sizes using particular scanning techniques, although in the general case one of ordinary skill recognizes that a variety of particular shapes and orientations and scanning methodologies may be used within the teaching of the present invention. A computer generally includes a processor for executing instructions and memory for storing instructions and data, including interfaces to obtain and process imaging data. When a general-purpose computer has a series of machine encoded instructions stored in its memory, the computer operating on such encoded instructions may become a specific type of machine, namely a computer particularly configured to perform the operations embodied by the series of instructions. Some of the instructions may be adapted to produce signals that control operation of other machines and thus may operate through those control signals to transform materials far removed from the computer itself. These descriptions and representations are the means used by those skilled in the art of data processing arts to most effectively convey the substance of their work to others skilled in the art.

An algorithm is here, and generally, conceived to be a self-consistent sequence of steps leading to a desired result. These steps are those requiring physical manipulations of physical quantities, observing and measuring scanned data representative of matter around the surgical site. Usually, though not necessarily, these quantities take the form of electrical or magnetic pulses or signals capable of being stored, transferred, transformed, combined, compared, and otherwise manipulated. It proves convenient at times, principally for reasons of common usage, to refer to these signals as bits, values, symbols, characters, display data, terms, numbers, or the like as a reference to the physical items or manifestations in which such signals are embodied or expressed to capture the underlying data of an image. It should be borne in mind, however, that all of these and similar terms are to be associated with the appropriate physical quantities and are merely used here as convenient labels applied to these quantities.

Some algorithms may use data structures for both inputting information and producing the desired result. Data structures greatly facilitate data management by data processing systems, and are not accessible except through sophisticated software systems. Data structures are not the information content of a memory, rather they represent specific electronic structural elements that impart or manifest a physical organization on the information stored in memory. More than mere abstraction, the data structures are specific electrical or magnetic structural elements in memory, which simultaneously represent complex data accurately, often data modeling physical characteristics of related items, and provide increased efficiency in computer operation.

Further, the manipulations performed are often referred to in terms, such as comparing or adding, commonly associated with mental operations performed by a human operator. No such capability of a human operator is necessary, or desirable in most cases, in any of the operations described herein that form part of the present invention; the operations are machine operations. Useful machines for performing the operations of the present invention include general-purpose digital computers or other similar devices. In all cases the distinction between the method operations in operating a computer and the method of computation itself should be recognized. The present invention relates to a method and apparatus for operating a computer in processing electrical or other (e.g., mechanical, chemical) physical signals to generate other desired physical manifestations or signals. The computer operates on software modules, which are collections of signals stored on a media that represents a series of machine instructions that enable the computer processor to perform the machine instructions that implement the algorithmic steps. Such machine instructions may be the actual computer code the processor interprets to implement the instructions, or alternatively may be a higher level coding of the instructions that is interpreted to obtain the actual computer code. The software module may also include a hardware component, wherein some aspects of the algorithm are performed by the circuitry itself rather as a result of an instruction.

The present invention also relates to an apparatus for performing these operations. This apparatus may be specifically constructed for the required purposes or it may comprise a general-purpose computer as selectively activated or reconfigured by a computer program stored in the computer. The algorithms presented herein are not inherently related to any particular computer or other apparatus unless explicitly indicated as requiring particular hardware. In some cases, the computer programs may communicate or relate to other programs or equipments through signals configured to particular protocols, which may or may not require specific hardware or programming to interact. In particular, various general-purpose machines may be used with programs written in accordance with the teachings herein, or it may prove more convenient to construct more specialized apparatus to perform the required method steps. The required structure for a variety of these machines will appear from the description below.

The present invention may deal with “object-oriented” software, and particularly with an “object-oriented” operating system. The “object-oriented” software is organized into “objects”, each comprising a block of computer instructions describing various procedures (“methods”) to be performed in response to “messages” sent to the object or “events” which occur with the object. Such operations include, for example, the manipulation of variables, the activation of an object by an external event, and the transmission of one or more messages to other objects. Often, but not necessarily, a physical object has a corresponding software object that may collect and transmit observed data from the physical device to the software system. Such observed data may be accessed from the physical object and/or the software object merely as an item of convenience; therefore where “actual data” is used in the following description, such “actual data” may be from the instrument itself or from the corresponding software object or module.

Messages are sent and received between objects having certain functions and knowledge to carry out processes. Messages are generated in response to user instructions, for example, by a user activating an icon with a “mouse” pointer generating an event. Also, messages may be generated by an object in response to the receipt of a message. When one of the objects receives a message, the object carries out an operation (a message procedure) corresponding to the message and, if necessary, returns a result of the operation. Each object has a region where internal states (instance variables) of the object itself are stored and where the other objects are not allowed to access. One feature of the object-oriented system is inheritance. For example, an object for drawing a “circle” on a display may inherit functions and knowledge from another object for drawing a “shape” on a display.

A programmer “programs” in an object-oriented programming language by writing individual blocks of code each of which creates an object by defining its methods. A collection of such objects adapted to communicate with one another by means of messages comprises an object-oriented program. Object-oriented computer programming facilitates the modeling of interactive systems in that each component of the system may be modeled with an object, the behavior of each component being simulated by the methods of its corresponding object, and the interactions between components being simulated by messages transmitted between objects.

An operator may stimulate a collection of interrelated objects comprising an object-oriented program by sending a message to one of the objects. The receipt of the message may cause the object to respond by carrying out predetermined functions, which may include sending additional messages to one or more other objects. The other objects may in turn carry out additional functions in response to the messages they receive, including sending still more messages. In this manner, sequences of message and response may continue indefinitely or may come to an end when all messages have been responded to and no new messages are being sent. When modeling systems utilizing an object-oriented language, a programmer need only think in terms of how each component of a modeled system responds to a stimulus and not in terms of the sequence of operations to be performed in response to some stimulus. Such sequence of operations naturally flows out of the interactions between the objects in response to the stimulus and need not be preordained by the programmer.

Although object-oriented programming makes simulation of systems of interrelated components more intuitive, the operation of an object-oriented program is often difficult to understand because the sequence of operations carried out by an object-oriented program is usually not immediately apparent from a software listing as in the case for sequentially organized programs. Nor is it easy to determine how an object-oriented program works through observation of the readily apparent manifestations of its operation. Most of the operations carried out by a computer in response to a program are “invisible” to an observer since only a relatively few steps in a program typically produce an observable computer output.

In the following description, several terms that are used frequently have specialized meanings in the present context. The term “object” relates to a set of computer instructions and associated data, which may be activated directly or indirectly by the user. The terms “windowing environment”, “running in windows”, and “object oriented operating system” are used to denote a computer user interface in which information is manipulated and displayed on a video display such as within bounded regions on a raster scanned video display. The terms “network”, “local area network”, “LAN”, “wide area network”, or “WAN” mean two or more computers that are connected in such a manner that messages may be transmitted between the computers. In such computer networks, typically one or more computers operate as a “server”, a computer with large storage devices such as hard disk drives and communication hardware to operate peripheral devices such as printers or modems. Other computers, termed “workstations”, provide a user interface so that users of computer networks may access the network resources, such as shared data files, common peripheral devices, and inter-workstation communication. Users activate computer programs or network resources to create “processes” which include both the general operation of the computer program along with specific operating characteristics determined by input variables and its environment. Similar to a process is an agent (sometimes called an intelligent agent), which is a process that gathers information or performs some other service without user intervention and on some regular schedule. Typically, an agent, using parameters typically provided by the user, searches locations either on the host machine or at some other point on a network, gathers the information relevant to the purpose of the agent, and presents it to the user on a periodic basis.

The term “desktop” means a specific user interface which presents a menu or display of objects with associated settings for the user associated with the desktop. When the desktop accesses a network resource, which typically requires an application program to execute on the remote server, the desktop calls an Application Program Interface, or “API”, to allow the user to provide commands to the network resource and observe any output. The term “Browser” refers to a program which is not necessarily apparent to the user, but which is responsible for transmitting messages between the desktop and the network server and for displaying and interacting with the network user. Browsers are designed to utilize a communications protocol for transmission of text and graphic information over a worldwide network of computers, namely the “World Wide Web” or simply the “Web”. Examples of Browsers compatible with the present invention include the Internet Explorer program sold by Microsoft Corporation (Internet Explorer is a trademark of Microsoft Corporation), the Opera Browser program created by Opera Software ASA, or the Firefox browser program distributed by the Mozilla Foundation (Firefox is a registered trademark of the Mozilla Foundation). Although the following description details such operations in terms of a graphic user interface of a Browser, the present invention may be practiced with text based interfaces, or even with voice or visually activated interfaces, that have many of the functions of a graphic based Browser.

Browsers display information, which is formatted in a Standard Generalized Markup Language (“SGML”) or a HyperText Markup Language (“HTML”), both being scripting languages, which embed non-visual codes in a text document through the use of special ASCII text codes. Files in these formats may be easily transmitted across computer networks, including global information networks like the Internet, and allow the Browsers to display text, images, and play audio and video recordings. The Web utilizes these data file formats to conjunction with its communication protocol to transmit such information between servers and workstations. Browsers may also be programmed to display information provided in an eXtensible Markup Language (“XML”) file, with XML files being capable of use with several Document Type Definitions (“DTD”) and thus more general in nature than SGML or HTML. The XML file may be analogized to an object, as the data and the stylesheet formatting are separately contained (formatting may be thought of as methods of displaying information, thus an XML file has data and an associated method).

The terms “personal digital assistant” or “PDA”, as defined above, means any handheld, mobile device that combines computing, telephone, fax, e-mail and networking features. The terms “wireless wide area network” or “WWAN” mean a wireless network that serves as the medium for the transmission of data between a handheld device and a computer. The term “synchronization” means the exchanging of information between a first device, e.g. a handheld device, and a second device, e.g. a desktop computer, either via wires or wirelessly. Synchronization ensures that the data on both devices are identical (at least at the time of synchronization).

In wireless wide area networks, communication primarily occurs through the transmission of radio signals over analog, digital cellular, or personal communications service (“PCS”) networks. Signals may also be transmitted through microwaves and other electromagnetic waves. At the present time, most wireless data communication takes place across cellular systems using second generation technology such as code-division multiple access (“CDMA”), time division multiple access (“TDMA”), the Global System for Mobile Communications (“GSM”), Third Generation (wideband or “3G”), Fourth Generation (broadband or “4G”), personal digital cellular (“PDC”), or through packet-data technology over analog systems such as cellular digital packet data (CDPD”) used on the Advance Mobile Phone Service (“AMPS”).

The terms “wireless application protocol” or “WAP” mean a universal specification to facilitate the delivery and presentation of web-based data on handheld and mobile devices with small user interfaces. “Mobile Software” refers to the software operating system, which allows for application programs to be implemented on a mobile device such as a mobile telephone or PDA. Examples of Mobile Software are Java and Java ME (Java and JavaME are trademarks of Sun Microsystems, Inc. of Santa Clara, Calif.), BREW (BREW is a registered trademark of Qualcomm Incorporated of San Diego, Calif.), Windows Mobile (Windows is a registered trademark of Microsoft Corporation of Redmond, Wash.), Palm OS (Palm is a registered trademark of Palm, Inc. of Sunnyvale, Calif.), Symbian OS (Symbian is a registered trademark of Symbian Software Limited Corporation of London, United Kingdom), ANDROID OS (ANDROID is a registered trademark of Google, Inc. of Mountain View, Calif.), and iPhone OS (iPhone is a registered trademark of Apple, Inc. of Cupertino, Calif.) , and Windows Phone 7. “Mobile Apps” refers to software programs written for execution with Mobile Software.

The terms “scan,” “fiducial reference”, “fiducial location”, “marker,” “tracker” and “image information” have particular meanings in the present disclosure. For purposes of the present disclosure, “scan” or derivatives thereof refer to x-ray, magnetic resonance imaging (MRI), computerized tomography (CT), sonography, cone beam computerized tomography (CBCT), or any system that produces a quantitative spatial representation of a patient. The term “fiducial reference” or simply “fiducial” refers to an object or reference on the image of a scan that is uniquely identifiable as a fixed recognizable point. In the present specification the term “fiducial location” refers to a useful location to which a fiducial reference is attached. A “fiducial location” will typically be proximate a surgical site. The term “marker” or “tracking marker” refers to an object or reference that may be perceived by a sensor proximate to the location of the surgical or dental procedure, where the sensor may be an optical sensor, a radio frequency identifier (RFID), a sonic motion detector, an ultra-violet or infrared sensor. The term “tracker” refers to a device or system of devices able to determine the location of the markers and their orientation and movement continually in ‘real time’ during a procedure. As an example of a possible implementation, if the markers are composed of printed targets then the tracker may include a stereo camera pair. The term “image information” is used in the present specification to describe information obtained by the tracker, whether optical or otherwise, and usable for determining the location of the markers and their orientation and movement continually in ‘real time’ during a procedure.

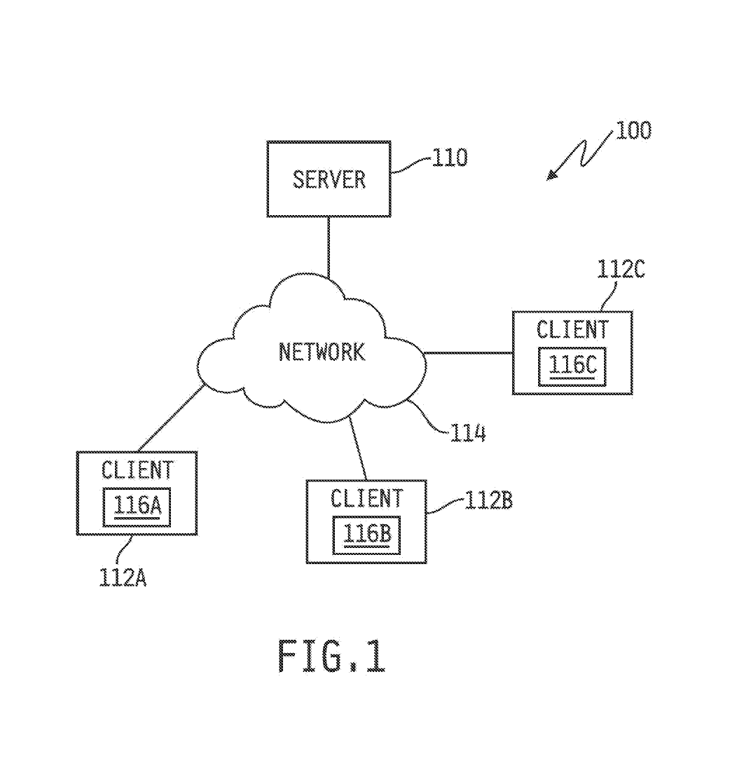

FIG. 1 is a high-level block diagram of a computing environment 100 according to one embodiment. FIG. 1 illustrates server 110 and three clients 112 connected by network 114. Only three clients 112 are shown in FIG. 1 in order to simplify and clarify the description. Embodiments of the computing environment 100 may have thousands or millions of clients 112 connected to network 114, for example the Internet. Users (not shown) may operate software 116 on one of clients 112 to both send and receive messages network 114 via server 110 and its associated communications equipment and software (not shown).

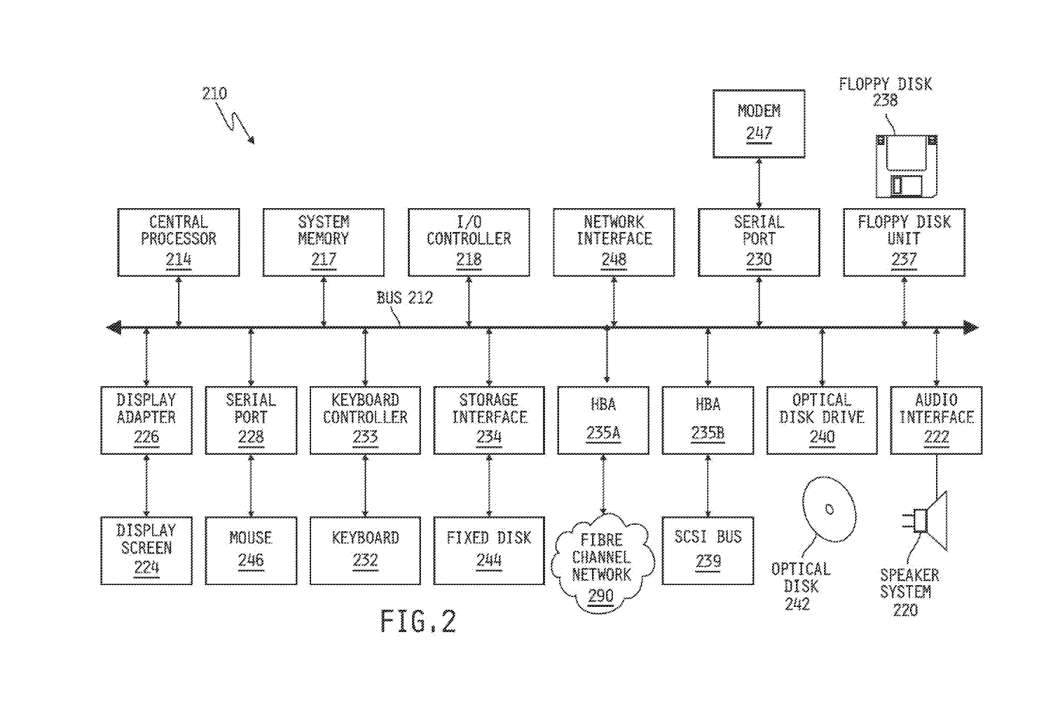

FIG. 2 depicts a block diagram of computer system 210 suitable for implementing server 110 or client 112. Computer system 210 includes bus 212 which interconnects major subsystems of computer system 210, such as central processor 214, system memory 217 (typically RAM, but which may also include ROM, flash RAM, or the like), input/output controller 218, external audio device, such as speaker system 220 via audio output interface 222, external device, such as display screen 224 via display adapter 226, serial ports 228 and 230, keyboard 232 (interfaced with keyboard controller 233), storage interface 234, disk drive 237 operative to receive floppy disk 238, host bus adapter (HBA) interface card 235A operative to connect with Fibre Channel network 290, host bus adapter (HBA) interface card 235B operative to connect to SCSI bus 239, and optical disk drive 240 operative to receive optical disk 242. Also included are mouse 246 (or other point-and-click device, coupled to bus 212 via serial port 228), modem 247 (coupled to bus 212 via serial port 230), and network interface 248 (coupled directly to bus 212).

Bus 212 allows data communication between central processor 214 and system memory 217, which may include read-only memory (ROM) or flash memory (neither shown), and random access memory (RAM) (not shown), as previously noted. RAM is generally the main memory into which operating system and application programs are loaded. ROM or flash memory may contain, among other software code, Basic Input-Output system (BIOS), which controls basic hardware operation such as interaction with peripheral components. Applications resident with computer system 210 are generally stored on and accessed via computer readable media, such as hard disk drives (e.g., fixed disk 244), optical drives (e.g., optical drive 240), floppy disk unit 237, or other storage medium. Additionally, applications may be in the form of electronic signals modulated in accordance with the application and data communication technology when accessed via network modem 247 or interface 248 or other telecommunications equipment (not shown).

Storage interface 234, as with other storage interfaces of computer system 210, may connect to standard computer readable media for storage and/or retrieval of information, such as fixed disk drive 244. Fixed disk drive 244 may be part of computer system 210 or may be separate and accessed through other interface systems. Modem 247 may provide direct connection to remote servers via telephone link or the Internet via an Internet service provider (ISP) (not shown). Network interface 248 may provide direct connection to remote servers via direct network link to the Internet via a POP (point of presence). Network interface 248 may provide such connection using wireless techniques, including digital cellular telephone connection, Cellular Digital Packet Data (CDPD) connection, digital satellite data connection or the like.

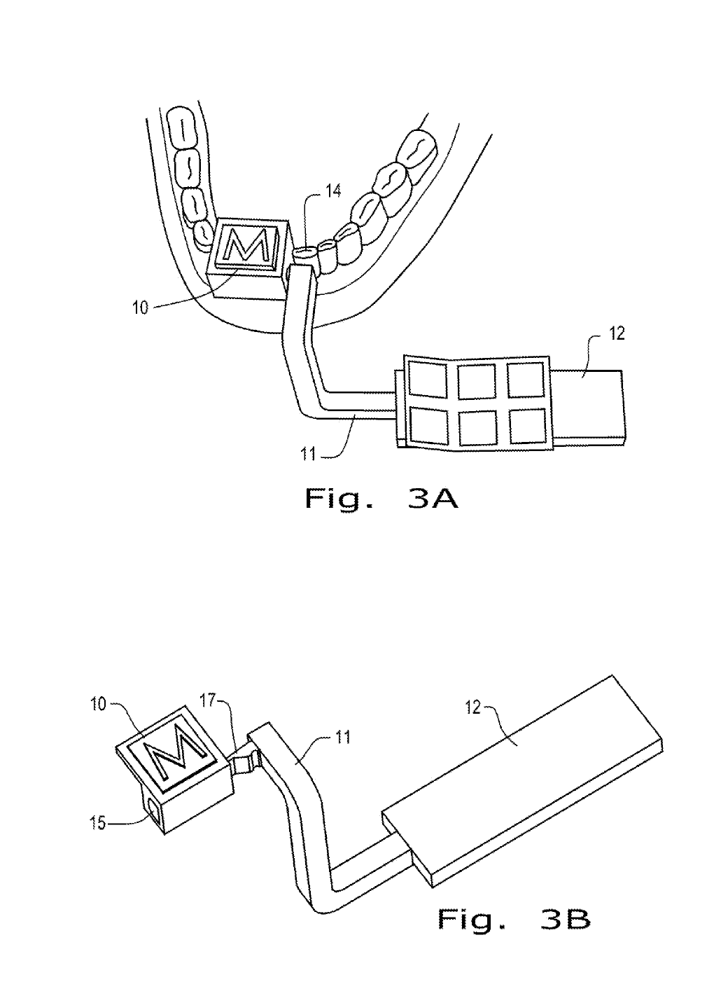

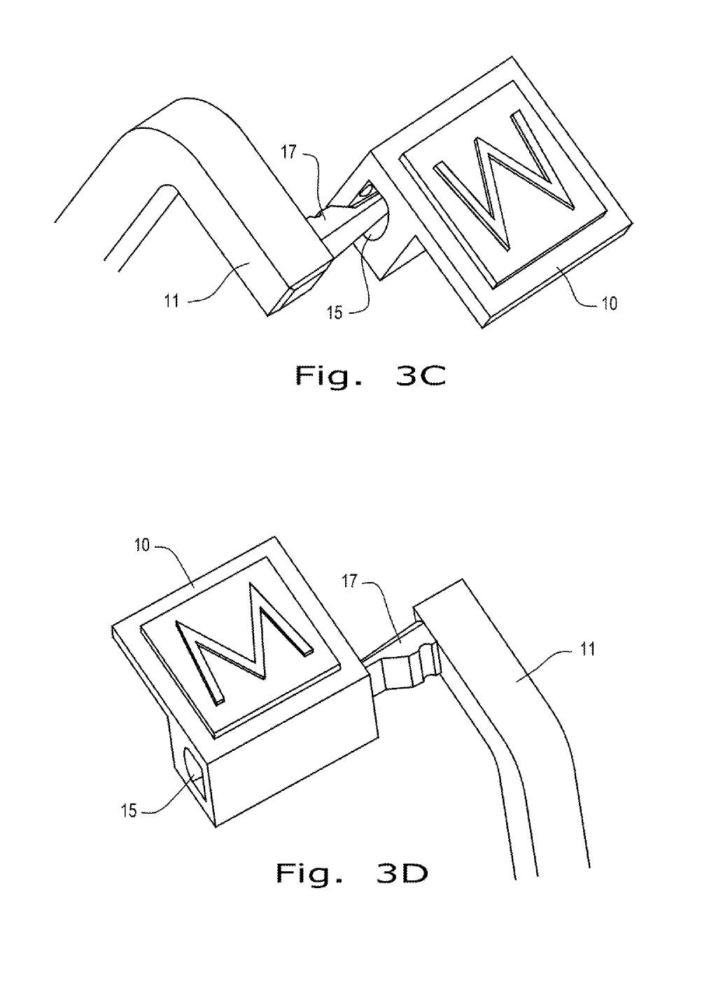

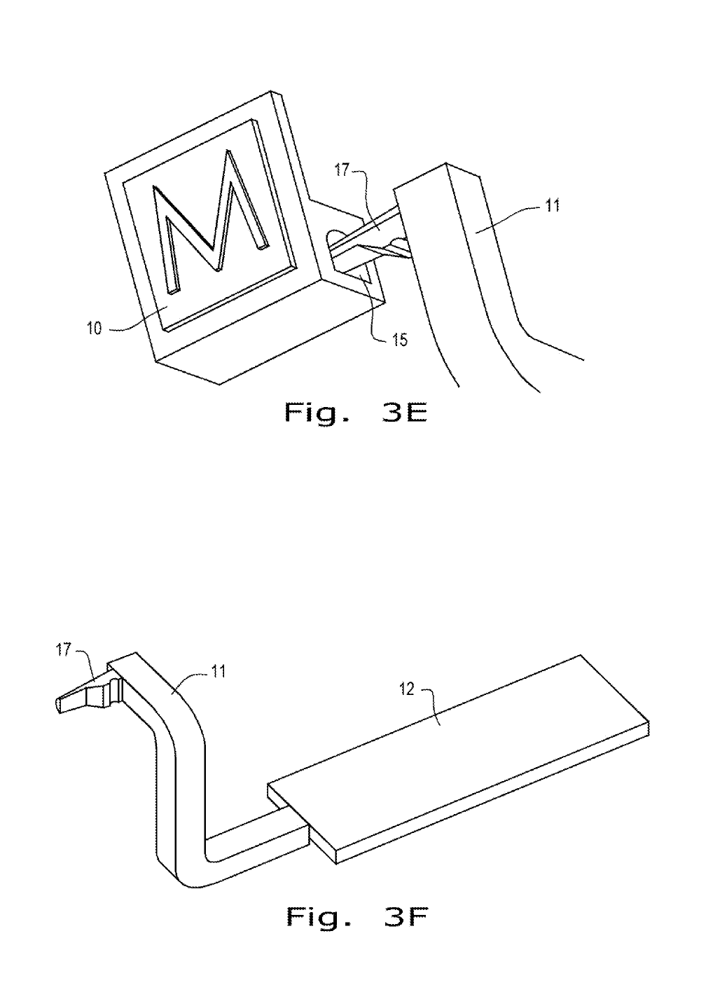

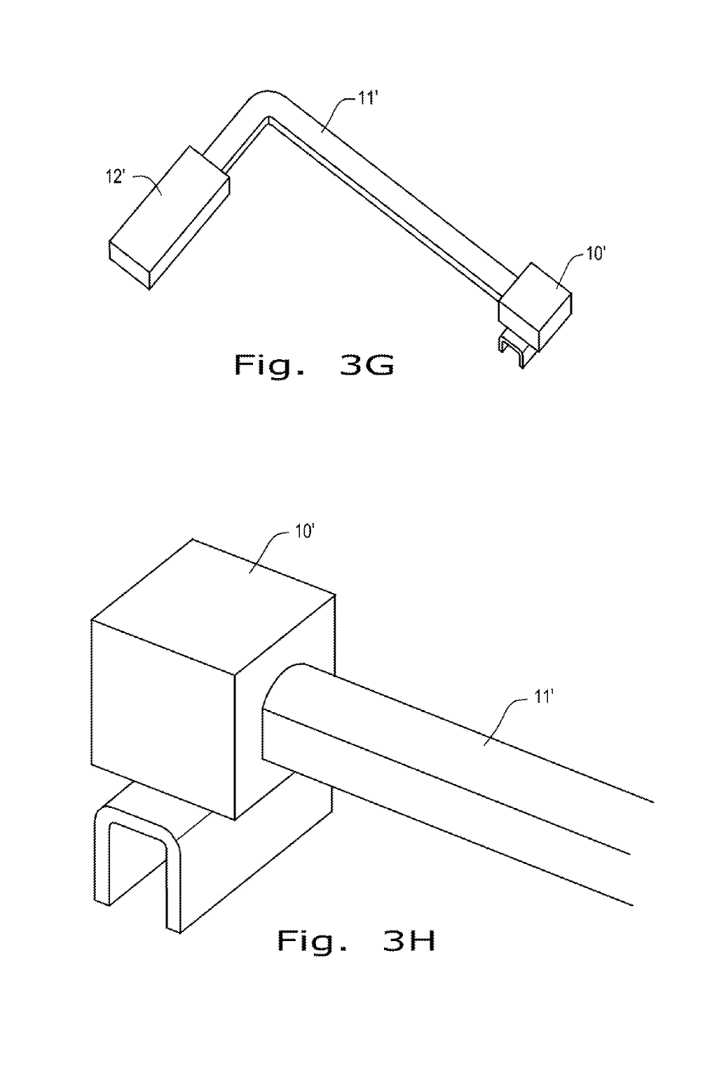

Many other devices or subsystems (not shown) may be connected in a similar manner (e.g., document scanners, digital cameras and so on), including the hardware components of FIGS. 3A-I, which alternatively may be in communication with associated computational resources through local, wide-area, or wireless networks or communications systems. Thus, while the disclosure may generally discuss an embodiment where the hardware components are directly connected to computing resources, one of ordinary skill in this area recognizes that such hardware may be remotely connected with computing resources. Conversely, all of the devices shown in FIG. 2 need not be present to practice the present disclosure. Devices and subsystems may be interconnected in different ways from that shown in FIG. 2. Operation of a computer system such as that shown in FIG. 2 is readily known in the art and is not discussed in detail in this application. Software source and/or object codes to implement the present disclosure may be stored in computer-readable storage media such as one or more of system memory 217, fixed disk 244, optical disk 242, or floppy disk 238. The operating system provided on computer system 210 may be a variety or version of either MS-DOS® (MS-DOS is a registered trademark of Microsoft Corporation of Redmond, Wash.), WINDOWS® (WINDOWS is a registered trademark of Microsoft Corporation of Redmond, Wash.), OS/2® (OS/2 is a registered trademark of International Business Machines Corporation of Armonk, N.Y.), UNIX® (UNIX is a registered trademark of X/Open Company Limited of Reading, United Kingdom), Linux® (Linux is a registered trademark of Linus Torvalds of Portland, Oreg.), or other known or developed operating system.

Moreover, regarding the signals described herein, those skilled in the art recognize that a signal may be directly transmitted from a first block to a second block, or a signal may be modified (e.g., amplified, attenuated, delayed, latched, buffered, inverted, filtered, or otherwise modified) between blocks. Although the signals of the above-described embodiments are characterized as transmitted from one block to the next, other embodiments of the present disclosure may include modified signals in place of such directly transmitted signals as long as the informational and/or functional aspect of the signal is transmitted between blocks. To some extent, a signal input at a second block may be conceptualized as a second signal derived from a first signal output from a first block due to physical limitations of the circuitry involved (e.g., there will inevitably be some attenuation and delay). Therefore, as used herein, a second signal derived from a first signal includes the first signal or any modifications to the first signal, whether due to circuit limitations or due to passage through other circuit elements which do not change the informational and/or final functional aspect of the first signal.

In co-pending patent applications PCT/IL2012/000363 and U.S. patent application Ser. No. 13/571,284, both expressly incorporated by reference herein, a surgical hardware and software monitoring system and associated method of use are described employing the fiducial key 10 and its associated tracking poles 11 and tracking markers 12. The present invention deploys a haptic device in conjunction with the system and method of co-pending patent application PCT/IL2012/000363 and U.S. patent application Ser. No. 13/571,284. For the sake of clarity, we address first the various embodiments of the surgical hardware and software monitoring system in the absence of the haptic device.

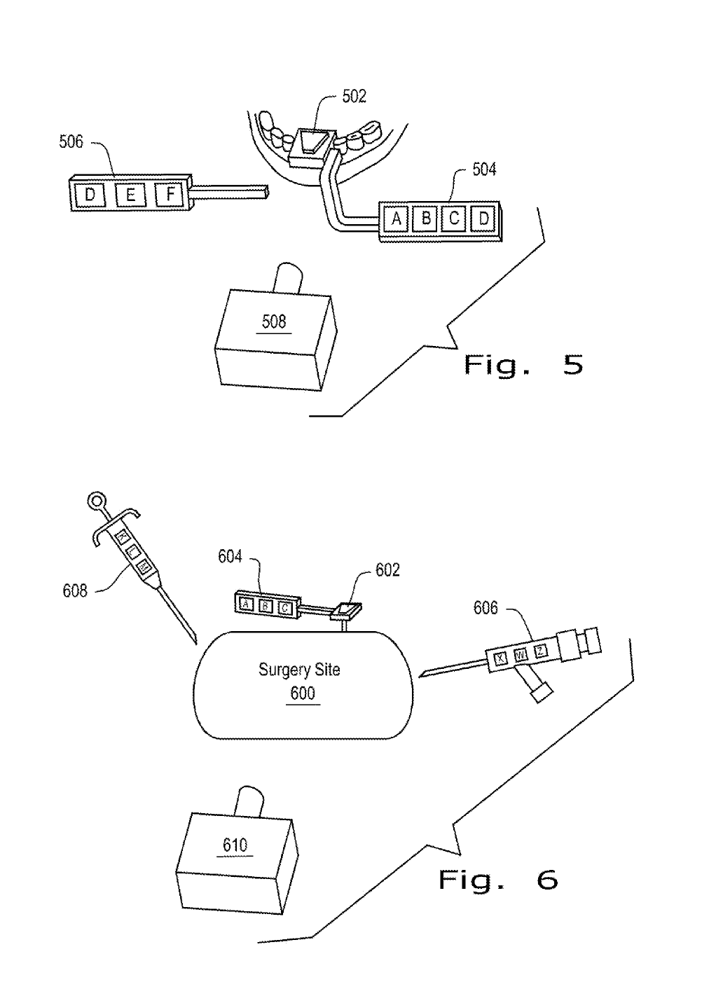

The surgical hardware and software monitoring system and method allow for surgical planning while the patient is available for surgery, for example while the patient is being prepared for surgery so that the system may model the surgical site. The system uses a particularly configured piece of hardware, represented as fiducial key 10 in FIG. 3A, to orient tracking marker 12 of the monitoring system with regard to the critical area of the surgery. Fiducial key 10 is attached to a location near the intended surgical area, in the exemplary embodiment of the dental surgical area of FIG. 3A, fiducial key 10 is attached to a dental splint 14. Tracking marker 12 may be connected to fiducial key 10 by tracking pole 11. In embodiments in which the fiducial reference is directly visible to a suitable tracker (see for example FIG. 5 and FIG. 6) that acquires image information about the surgical site, a tracking marker may be attached directly to the fiducial reference. For example a dental surgery, dental tracking marker 14 may be used to securely locate the fiducial 10 near the surgical area. Fiducial key 10 may be used as a point of reference, or a fiducial, for the further image processing of data acquired from tracking marker 12 by the tracker.

In other embodiments additional tracking markers 12 may be attached to items independent of the fiducial key 10 and any of its associated tracking poles 11 or tracking markers 12. This allows the independent items to be tracked by the tracker.

In a further embodiment at least one of the items or instruments near the surgical site may optionally have a tracker attached to function as tracker for the monitoring system of the invention and to thereby sense the orientation and the position of tracking marker 12 and of any other additional tracking markers relative to the scan data of the surgical area. By way of example, the tracker attached to an instrument may be a miniature digital camera and it may be attached, for example, to a dentist's drill. Any other markers to be tracked by the tracker attached to the item or instrument must be within the field of view of the tracker.

Using the dental surgery example, the patient is scanned to obtain an initial scan of the surgical site. The particular configuration of fiducial key 10 allows computer software stored in memory and executed in a suitable controller, for example processor 214 and memory 217 of computer 210 of FIG. 2, to recognize its relative position within the surgical site from the scan data, so that further observations may be made with reference to both the location and orientation of fiducial key 10. In some embodiments, the fiducial reference includes a marking that is apparent as a recognizable identifying symbol when scanned. In other embodiments, the fiducial reference includes a shape that is distinct in the sense that the body apparent on the scan has an asymmetrical form allowing the front, rear, upper, and lower, and left/right defined surfaces that may be unambiguously determined from the analysis of the scan, thereby to allow the determination not only of the location of the fiducial reference, but also of its orientation.

In addition, the computer software may create a coordinate system for organizing objects in the scan, such as teeth, jaw bone, skin and gum tissue, other surgical instruments, etc. The coordinate system relates the images on the scan to the space around the fiducial and locates the instruments bearing markers both by orientation and position. The model generated by the monitoring system may then be used to check boundary conditions, and in conjunction with the tracker, display the arrangement in real time on a suitable display, for example display 224 of FIG. 2.

In one embodiment, the computer system has a predetermined knowledge of the physical configuration of fiducial key 10 and examines slices/sections of the scan to locate fiducial key 10. Locating fiducial key 10 may be on the basis of its distinct shape, or on the basis of distinctive identifying and orienting markings upon fiducial key 10 or on attachments to fiducial key 10 such as tracking marker 12. Fiducial key 10 may be rendered distinctly visible in the scans through higher imaging contrast by employing radio-opaque materials or high-density materials in the construction of the fiducial key 10. In other embodiments the material of the distinctive identifying and orienting markings may be created using suitable high density or radio-opaque inks or materials.

Once fiducial key 10 is identified, the location and orientation of fiducial key 10 is determined from scan segments, and a point within fiducial key 10 is assigned as the center of the coordinate system. The point so chosen may be chosen arbitrarily, or the choice may be based on some useful criterion. A model is then derived, for example in the form of a transformation matrix, to relate the fiducial system, being fiducial key 10 in one particular embodiment, to the coordinate system of the surgical site. The resulting virtual construct may be used by surgical procedure planning software for virtual modeling of the contemplated procedure, and may alternatively be used by instrumentation software for the configuration of the instrument, for providing imaging assistance for surgical software, and/or for plotting trajectories for the conduct of the surgical procedure.

In some embodiments, the monitoring hardware includes a tracking attachment to the fiducial reference. In the embodiment pertaining to dental surgery the tracking attachment to fiducial key 10 is tracking marker 12, which is attached to fiducial key 10 via tracking pole 11. Tracking marker 12 may have a particular identifying pattern. The trackable attachment, for example tracking marker 12, and even associated tracking pole 11 may have known configurations so that observational data from tracking pole 11 and/or tracking marker 12 may be precisely mapped to the coordinate system, and thus progress of the surgical procedure may be monitored and recorded. For example, as particularly shown in FIG. 3J, fiducial key 10 may have hole 15 in a predetermined location specially adapted for engagement with insert 17 of tracking pole 11. In such an arrangement, for example, tracking poles 11 may be attached with a low force push into hole 15 of fiducial key 10, and an audible haptic notification may thus be given upon successful completion of the attachment.

It is further possible to reorient tracking pole 11 during a surgical procedure. Such reorientation may be in order to change the location of the procedure, for example where a dental surgery deals with teeth on the opposite side of the mouth, where a surgeon switches hands, and/or where a second surgeon performs a portion of the procedure. For example, the movement of tracking pole 11 may trigger a re-registration of tracking pole 11 with relation to the coordinate system, so that the locations may be accordingly adjusted. Such a re-registration may be automatically initiated when, for example in the case of the dental surgery embodiment, tracking pole 11 with its attached tracking marker 12 is removed from hole 15 of fiducial key 10 and another tracking marker with its associated tracking pole is connected to an alternative hole on fiducial key 10. Additionally, boundary conditions may be implemented in the software so that the user is notified when observational data approaches and/or enters the boundary areas, for example by an audio, haptic, and/or visual indication.

In a further embodiment of the system, a surgical instrument or implement, herein termed a “hand piece” (see FIGS. 5 and 6), may also have a particular configuration that may be located and tracked in the coordinate system and may have suitable tracking markers as described herein. A boundary condition may be set up to indicate a potential collision with virtual material, so that when the hand piece is sensed to approach the boundary condition an indication may appear on a screen, a haptic feedback may be provided, or an alarm may sound. Further, target boundary conditions may be set up to indicate the desired surgical area, so that when the trajectory of the hand piece is trending outside the target area an indication may appear on screen, a haptic feedback may be provided, or an alarm sound indicating that the hand piece is deviating from its desired path.

An alternative embodiment of some hardware components is shown in FIGS. 3G-I. Fiducial key 10′ has connection elements with suitable connecting portions to allow a tracking pole 11′ to position a tracking marker 12′ relative to the surgical site. Conceptually, fiducial key 10′ serves as an anchor for pole 11′ and tracking marker 12′ in much the same way as the earlier embodiment, although it has a distinct shape. The software of the monitoring system is pre-programmed with the configuration of each particularly identified fiducial key, tracking pole, and tracking marker, so that the location calculations are only changed according to the changed configuration parameters.

The materials of the hardware components may vary according to regulatory requirements and practical considerations the key or fiducial component 10, 10′ may be made of generally radio opaque material such that it does not produce noise for the scan, yet creates recognizable contrast on the scanned image so that any identifying pattern associated with it may be recognized. In addition, because it is generally located on the patient, the material should be lightweight and suitable for connection to an apparatus on the patient. For example, in the dental surgery example, the materials of the fiducial key 10, 10′ must be suitable for connection to a plastic splint and suitable for connection to a tracking pole. In the surgical example the materials of the fiducial key 10, 10′ may be suitable for attachment to skin, bones, teeth, or other particular body tissue of a patient.

Tracking markers 12, 12′ are clearly identified by employing, for example without limitation, high contrast pattern engraving. The materials of tracking markers 12, 12′ are chosen to be capable of resisting damage in autoclave processes and are compatible with rigid, repeatable, and quick connection to a connector structure. Tracking markers 12, 12′ and associated tracking poles 11, 11′ have the ability to be accommodated at different locations for different surgery locations, and, like fiducial keys 10, 10′, they should also be relatively lightweight as they will often be resting on or against the patient. Tracking poles 11, 11′ must similarly be compatible with autoclave processes and have connectors of a form shared among tracking poles 11, 11′.

The tracker employed in tracking fiducial keys 10, 10′, tracking poles 11, 11′ and tracking markers 12, 12′ should be capable of tracking with suitable accuracy objects of a size of the order of 1.5 square centimeters. The tracker may be, by way of example without limitation, a stereo camera or stereo camera pair. While the tracker is generally connected by wire to a computing device to read the sensory input, it may optionally have wireless connectivity to transmit the sensory data to a computing device.

In embodiments that additionally employ a trackable piece of instrumentation, such as a hand piece, tracking markers attached to such a trackable piece of instrumentation may also be light-weight; capable of operating in a 3 object array with 90 degrees relationship; optionally having a high contrast pattern engraving and a rigid, quick mounting mechanism to a standard hand piece.

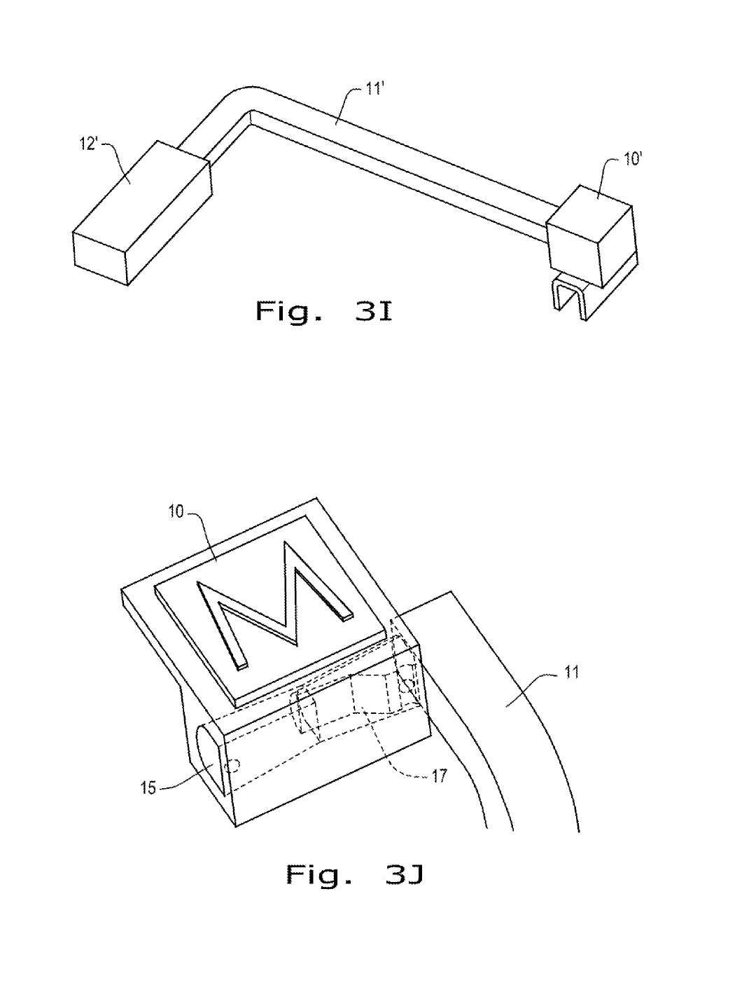

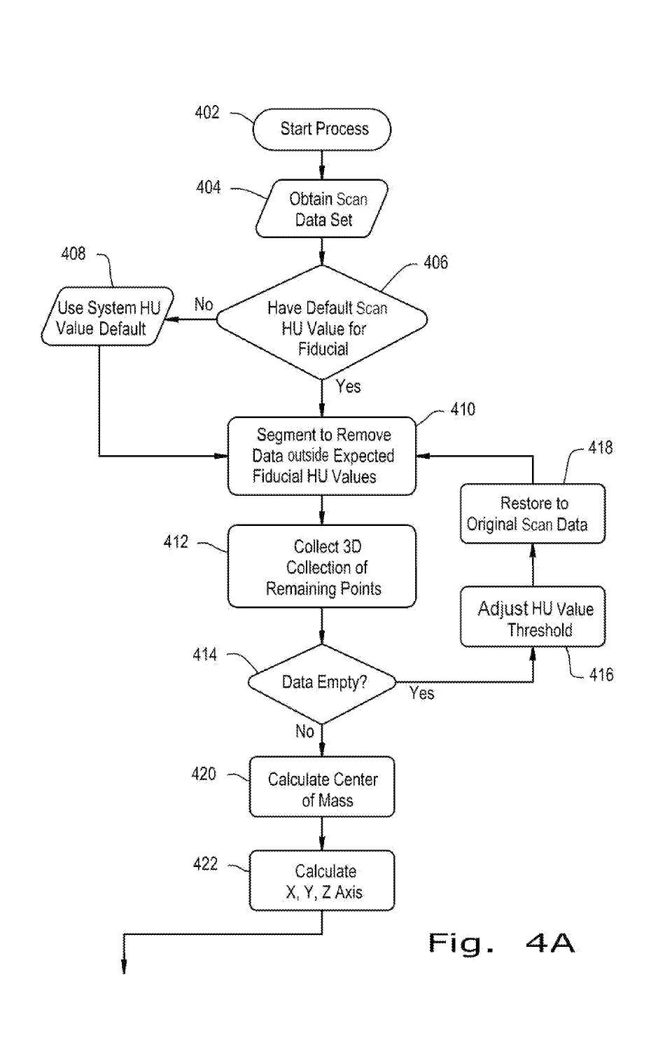

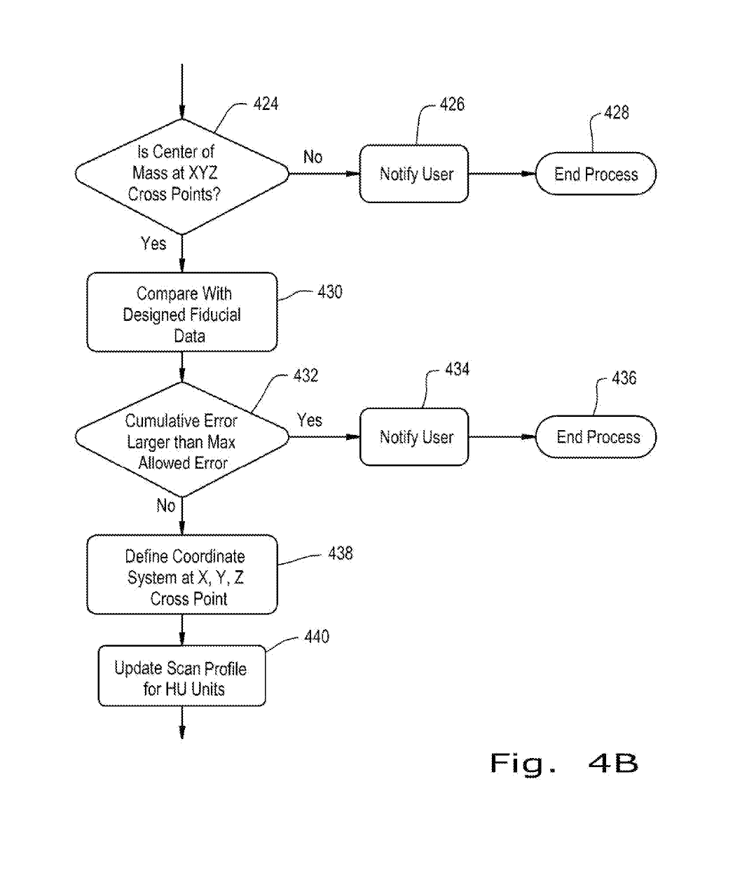

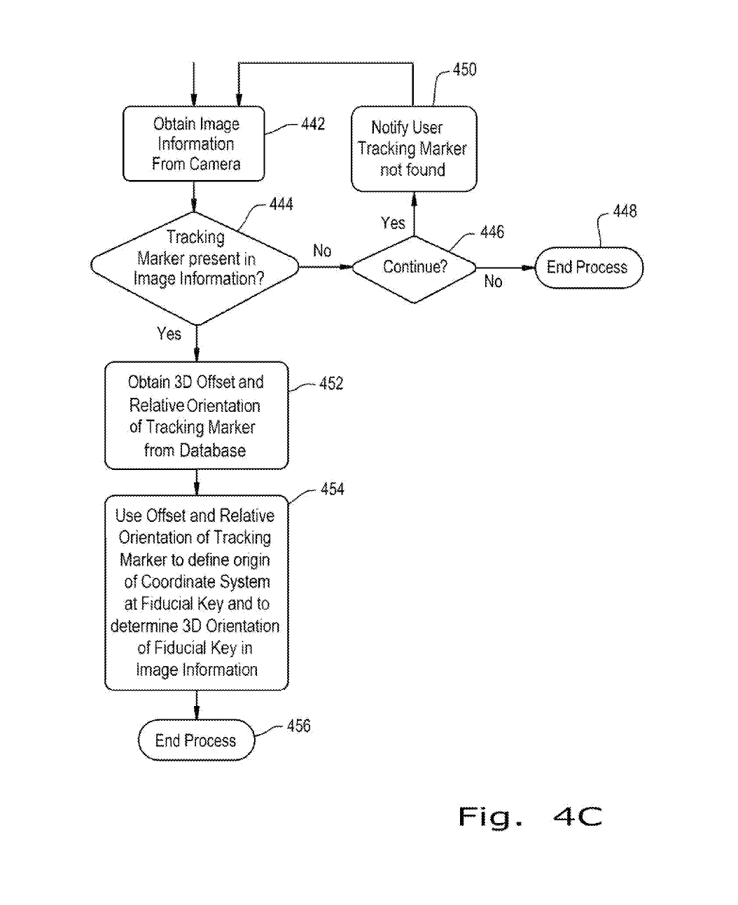

Another aspect of the invention involves an automatic registration method for tracking surgical activity, as illustrated in FIGS. 4A-C. FIG. 4A and FIG. 4B together present, without limitation, a flowchart of one method for determining the three-dimensional location and orientation of the fiducial reference from scan data. FIG. 4C presents a flow chart of a method for confirming the presence of a suitable tracking marker in image information obtained by the tracker and determining the three-dimensional location and orientation of the fiducial reference based on the image information.

Once the process starts [402], as described in FIGS. 4A and 4B, the system obtains a scan data set [404] from, for example, a CT scanner and checks for a default CT scan Hounsfield unit (HU) value [at 406] for the fiducial which may or may not have been provided with the scan based on a knowledge of the fiducial and the particular scanner model, and if such a threshold value is not present, then a generalized predetermined default value is employed [408]. Next the data is processed by removing scan segments with Hounsfield data values outside expected values associated with the fiducial key values [at 410], following the collection of the remaining points [at 412]. If the data is empty [at 414], the CT value threshold is adjusted [at 416], the original value restored [at 418], and the segmenting processing scan segments continues [at 410]. Otherwise, with the existing data a center of mass is calculated [at 420], along with calculating the X, Y, and Z axes [at 422]. If the center of mass is not at the cross point of the XYZ axes [at 424], then the user is notified [at 426] and the process stopped [at 428]. If the center of mass is at the XYZ cross point then the data points are compared with designed fiducial data [430]. If the cumulative error is larger than the maximum allowed error [432] then the user is notified [at 434] and the process ends [at 436]. If not, then the coordinate system is defined at the XYZ cross point [at 438], and the scan profile is updated for the HU units [at 440].

Turning now to FIG. 4C, an image is obtained from the tracker, being a suitable camera or other sensor [442]. The image information is analyzed to determine whether a tracking marker is present in the image information [444]. If not, then the user is queried [466] as to whether the process should continue or not. If not, then the process is ended

If the process is to continue, then the user may be notified that no tracking marker has been found in the image information [450], and the process returns to obtaining image information [442]. If a tracking marker has been found based on the image information, or one has been attached by the user upon the above notification [450], the offset and relative orientation of the tracking marker to the fiducial reference is obtained from a suitable database [452]. The term “database” is used in this specification to describe any source, amount or arrangement of such information, whether organized into a formal multi-element or multi-dimensional database or not. A single data set comprising offset value and relative orientation may suffice in some embodiments and may be provided, for example, by the user or may be within a memory unit of the controller or in a separate database or memory.

The offset and relative orientation of the tracking marker is used to define the origin of a coordinate system at the fiducial reference and to determine the three-dimensional orientation of the fiducial reference based on the image information [454] and the registration process ends [458]. In order to monitor the location and orientation of the fiducial reference in real time, the process may be looped back from step [454] to obtain new image information from the camera [442]. A suitable query point may be included to allow the user to terminate the process. Detailed methods for determining orientations and locations of predetermined shapes or marked tracking markers from image data are known to practitioners of the art and will not be dwelt upon here. The coordinate system so derived is then used for tracking the motion of any items bearing tracking markers in the proximity of the surgical site. Other registration systems are also contemplated, for example using current other sensory data rather than the predetermined offset, or having a fiducial with a transmission capacity.

One example of an embodiment is shown in FIG. 5. In addition to fiducial key 502 mounted at a predetermined tooth and having a rigidly mounted tracking marker 504, an additional instrument or implement 506, for example a hand piece which may be a dental drill, may be observed by a camera 508 serving as tracker of the monitoring system.

Another example embodiment is shown in FIG. 6. Surgery site 600, for example a human stomach or chest, may have fiducial key 602 fixed to a predetermined position to support tracking marker 604. Endoscope 606 may have further tracking markers, and biopsy needle 608 may also be present bearing a tracking marker at surgery site 600. Sensor 610, may be for example a camera, infrared sensing device, or RADAR.

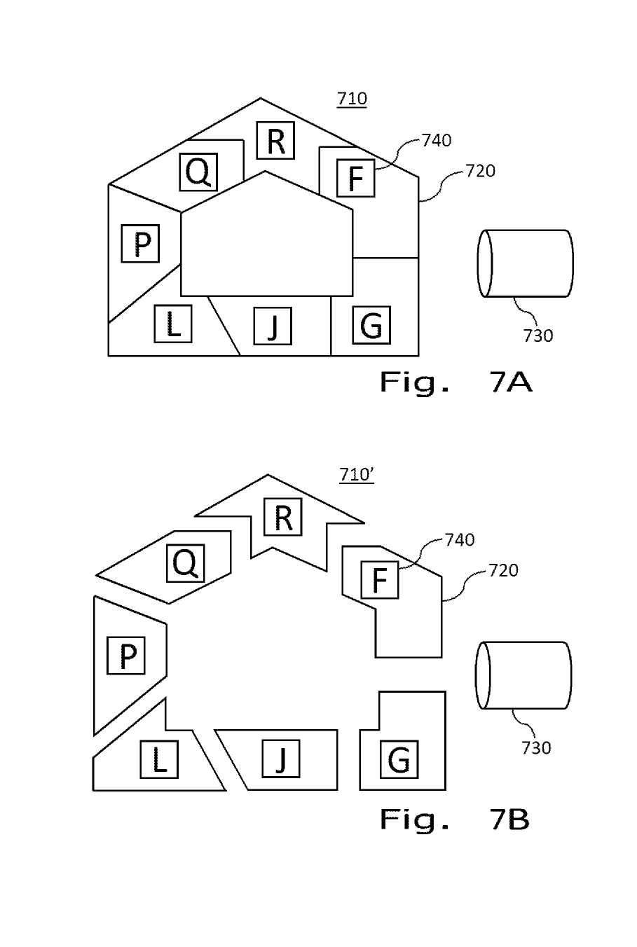

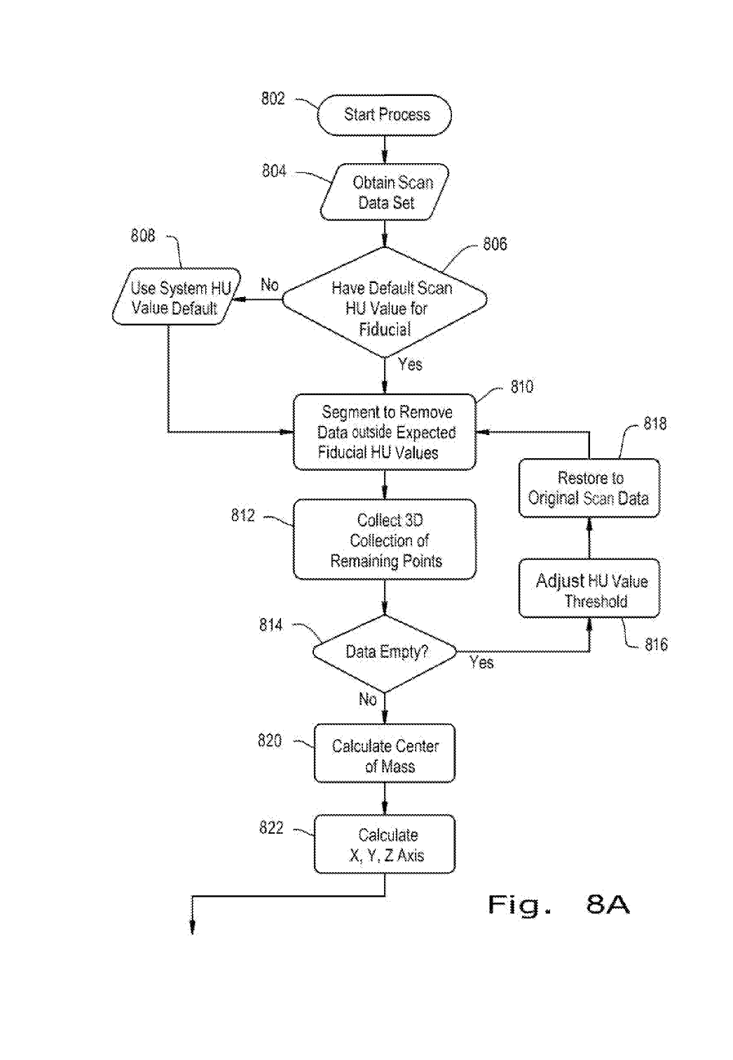

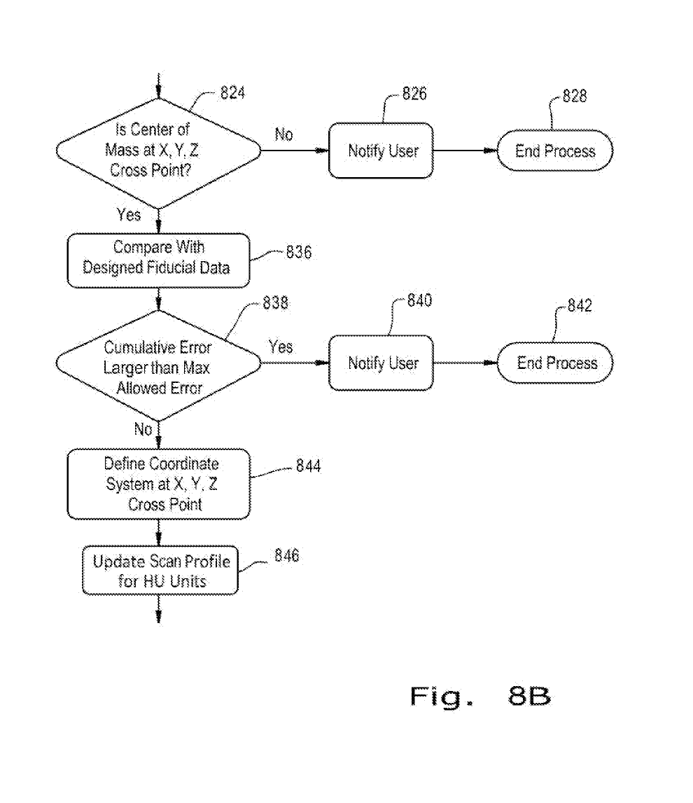

In another embodiment of the surgical monitoring system, shown schematically in FIG. 7A, the fiducial key may comprise a multi-element fiducial pattern 710. In one implementation the multi-element fiducial pattern 710 may be a dissociable pattern. The term “dissociable pattern” is used in this specification to describe a pattern comprising a plurality of pattern segments 720 that topologically fit together to form a contiguous whole pattern, and which may temporarily separated from one another, either in whole or in part. The term “breakable pattern” is used as an alternative term to describe such a dissociable pattern. In other embodiments, the segments of multi-element fiducial pattern 710 do not form a contiguous pattern, but instead their positions and orientations with respect to one another are known when multi-element fiducial pattern 710 is applied on the body of the patient near a critical area of a surgical site. Each pattern segment 720 is individually locatable based on scan data of a surgical site to which multi-element fiducial pattern 710 may be attached.

Pattern segments 720 are uniquely identifiable by suitable tracker 730, being differentiated from one another in one or more of a variety of ways. Pattern segments 720 may be mutually differentiable shapes that also allow the identification of their orientations. Pattern segments 720 may be uniquely marked in one or more of a variety of ways, including but not limited to barcoding or orientation-defining symbols. The marking may be directly on pattern segments 720, or may be on tracking markers 740 attached to pattern segments 720. Marking may be accomplished by a variety of methods, including but not limited to engraving and printing. In the embodiment shown in FIGS. 7A and 7B, by way of non-limiting example, the letters F, G, J, L, P, Q and R have been used.

The materials of multi-element fiducial pattern 710 and pattern segments 720, and of any tracking markers 740 attached to them, may vary according to regulatory requirements and practical considerations. Generally, the key or fiducial component is made of generally radio opaque material such that it does not produce noise for the scan, yet creates recognizable contrast on the scanned image so that any identifying pattern associated with it may be recognized. Multi-element fiducial pattern 710 and pattern segments 720 may have a distinct coloration difference from human skin in order to be more clearly differentiable by tracker 730. In addition, because it is generally located on the patient, the material should be lightweight. The materials should also be capable of resisting damage in autoclave processes.

A suitable tracker of any of the types already described is used to locate and image multi-element fiducial pattern 710 within the surgical area. Multi-element fiducial pattern 710 may be rendered distinctly visible in scans of the surgical area through higher imaging contrast by the employ of radio-opaque materials or high-density materials in the construction of the multi-element fiducial pattern 710. In other embodiments the distinctive identifying and orienting markings on the pattern segments 720 or on the tracking markers 740 may be created using suitable high-density materials or radio-opaque inks, thereby allowing the orientations of pattern segments 720 to be determined based on scan data.

During surgery the surgical area may undergo changes in position and orientation. This may occur, for example, as a result of the breathing or movement of the patient. In this process, as shown in FIG. 7B, pattern segments 720 of multi-element fiducial pattern 710 may change their relative locations and also, in general, their relative orientations. Information on these changes may be used to gain information on the subcutaneous motion of the body of the patient in the general vicinity of the surgical site by relating the changed positions and orientations of pattern segments 720 to their locations and orientations in a scan done before surgery. In this sense, while pattern segments 720 may be associated with a particular portion of the surgical site, while in another sense pattern segments move with movement of tissue in the surgical site, and thus may be said to move with the surgical site.

Using abdominal surgery as example, the patient is scanned, for example by an x-ray, magnetic resonance imaging (MRI), computerized tomography (CT), or cone beam computerized tomography (CBCT), to obtain an initial image of the surgical site. The particular configuration of multi-element fiducial pattern 710 allows computer software to recognize its relative position within the surgical site, so that further observations may be made with reference to both the location and orientation of multi-element fiducial pattern 710. In fact, the computer software may create a coordinate system for organizing objects in the scan, such as skin, organs, bones, and other tissue, other surgical instruments bearing suitable tracking markers, and segments 720 of multi-element fiducial pattern 710 etc.

In one embodiment, the computer system has a predetermined knowledge of the configuration of multi-element fiducial pattern 710 and examines slices of a scan of the surgical site to locate pattern segments 720 of multi-element fiducial pattern 710 based on one or more of the radio-opacity density of the material of pattern segments 720, their shapes and their unique tracking markers 740. Once the locations and orientations of pattern segments 720 have been determined, a point within or near multi-element fiducial pattern 710 is assigned as the center of the coordinate system. The point so chosen may be chosen arbitrarily, or the choice may be based on some determined criteria based on the type of surgery, type of instrumentation, etc. A transformation matrix is derived to relate multi-element fiducial pattern 710 to the coordinate system of the surgical site. The resulting virtual construct may then be used by surgical procedure planning software for virtual modeling of the contemplated procedure, and may alternatively be used by instrumentation software for the configuration of the instrument, for providing imaging assistance for surgical software, and/or for plotting trajectories for the conduct of the surgical procedure.

Multi-element fiducial pattern 710 may change its shape as portions of the body tissue moves during surgery. The relative locations and relative orientations of pattern segments 720 may thus change in the process. (see FIG. 7A relative to FIG. 7B.) In this process the integrity of individual pattern segments 720 is maintained and they may be tracked by tracker 730, including but not limited to a stereo video camera. The changed orientation of multi-element fiducial pattern 710′ may be compared with initial orientation of multi-element fiducial pattern 710′ to create a transformation matrix. The relocating and reorienting of pattern segments 720 may therefore be mapped on a continuous basis within the coordinate system of the surgical site. In FIGS. 7A and 7B a total of seven pattern segments 720 are shown. In other embodiments multi-element fiducial pattern 710 may comprise larger or smaller numbers of pattern segments 720. During operation of the surgical monitoring system of this embodiment a selection of pattern segments 720 may be employed and there is no requirement that all pattern segments 720 of multi-element fiducial pattern 710 must be employed. The decision as to how many pattern segments 720 to employ may, by way of example, be based on the resolution required for the surgery to be performed or on the processing speed of the controller, which may be, for example, computer 210 of FIG. 2.

For the sake of clarity, FIG. 7A employs a dissociable multi-element fiducial pattern. In other embodiments the multi-element fiducial pattern may have a dissociated fiducial pattern, such as that of FIG. 7B, as default. Individual pattern segments 720 then may change position as the body tissue of the patient changes orientation or shape near the surgical site during the surgery. In yet other embodiments tracking markers 740 may be absent and the tracking system may rely on tracking pattern segments 720 purely on the basis of their unique shapes, which lend themselves to determining orientation due to a lack of a center of symmetry. As already pointed out, in other embodiments pattern segments 720 are not in general limited to being capable of being joined topologically at their perimeters to form a contiguous surface. Nor is there a particular limitation on the general shape of multi-element fiducial pattern 710.

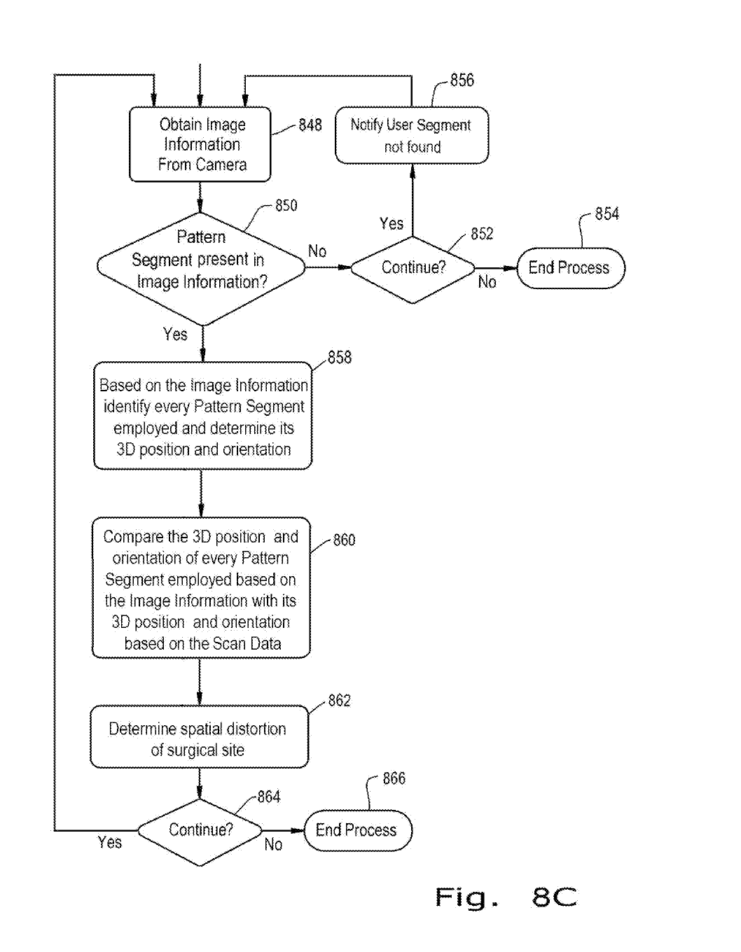

An automatic registration method for tracking surgical activity using multi-element fiducial pattern 710 is shown in the flow chart diagram of FIG. 8A, FIG. 8B and FIG. 8C. FIG. 8A and FIG. 8B together present, without limitation, a flowchart of one method for determining the three-dimensional location and orientation of one segment of multi-element fiducial pattern 710 from scan data. FIG. 8C presents a flow chart of a method for determining the spatial distortion of the surgical site based on the changed orientations and locations of pattern segments 720 of multi-element fiducial pattern 710, using as input the result of applying the method shown in FIG. 8A and FIG. 8B to every one of pattern segments 720 that is to be employed in the determining the spatial distortion of the surgical site. In principle, not all pattern segments 720 need to be employed.

Once the process starts [802], as described in FIGS. 8A and 8B, the system obtains scan data set [404] from, for example, a CT scanner and checks for a default CT scan Hounsfield unit (HU) value [806] for the fiducial, which may or may not have been provided with the scan based on a knowledge of the fiducial and the particular scanner model. If such a default value is not present, then a generalized predetermined system default value is employed [808]. Next the data is processed by removing scan slices or segments with Hounsfield data values outside the expected values associated with the fiducial key [810], followed by the collecting of the remaining points [812]. If the data is empty [814], the CT value threshold is adjusted [816], the original data restored [818], and the processing of scan slices continues [810]. Otherwise, with the existing data a center of mass is calculated [820], as are the X, Y and Z axes [822]. If the center of mass is not at the X, Y, Z cross point [824], then the user is notified [826] and the process ended [828]. If the center of mass is at the X, Y, Z cross point [824], then the pattern of the fiducial is compared to the data [836], and if the cumulative error is larger than the maximum allowed error [838] the user is notified [840] and the process is ended [842]. If the cumulative error is not larger than the maximum allowed error [838], then the coordinate system is defined at the XYZ cross-point [844] and the CT profile is updated for HU units [846]. This process of FIG. 8A and FIG. 8B is repeated for every one of pattern segments 720 that is to be employed in determining the spatial distortion of the surgical site. The information on the location and orientation of every one of pattern segments 720 is then used as input to the method described relating to FIG. 8C.

Turning now to FIG. 8C, image information is obtained from the camera [848] and it is determined whether any particular segment 720 of multi-element fiducial pattern 710 on the patient body is present in the image information [850]. If no particular segment 720 is present in the image information, then the user is queried as to whether the process should continue [852]. If not, then the process is ended [854]. If the process is to continue, the user is notified that no particular segment 720 was not found in the image information [856] and the process returns to obtaining image information from the camera [848]. If one of particular segments 720 is present in the image information at step [850], then, every other pattern segment 720 employed is identified and the three-dimensional location and orientation of all segments 720 employed are determined based on the image information [858]. The three-dimensional location and orientation of every pattern segment employed based on the image information is compared with the three dimensional location and orientation of the same pattern segment as based on the scan data [860]. Based on this comparison the spatial distortion of the surgical site is determined [862]. In order to monitor such distortions in real time, the process may be looped back to obtain image information from the camera [848]. A suitable query point [864] may be included to allow the user to terminate the process [866]. Detailed methods for determining orientations and locations of predetermined shapes or marked tracking markers from image data are known to practitioners of the art and will not be dwelt upon here.

By the above method the software of the controller, for example computer 210 of FIG. 2, is capable of recognizing multi-element fiducial pattern 710 and calculating a model of the surgical site based on the identity of multi-element fiducial pattern 710 and its changes in shape, orientation, or configuration based on the observation data received from multi-element fiducial pattern 710. This allows the calculation in real time of the locations and orientations of anatomical features in the proximity of multi-element fiducial pattern 710.

In a further aspect of the surgical monitoring system and method described in the foregoing, a haptic sensory feedback device is employed in conjunction with the model generated by the monitoring system from the scan data of the surgical site. This allows the user, who may be a surgeon, to “virtually touch” or interact with the virtual items and materials in the model, the virtual touching or interacting taking place safely in virtual space. For example, in dental surgery a virtual drill may be manipulated in virtual space by the user via the haptic device. The virtual drill may touch a virtual item in the virtual 3D representation of the surgical site and the appropriate resistance or reactive force may be created and imposed as feedback via the haptic device on the user. The resistance or reactive force is based on the particular virtual material, for example bone, gum or cheek, and is imposed on the user-via the haptic device, providing physical sensory haptic feedback to the user. The surgical hardware and software monitoring system of the present invention may be configured to provide a user selectable choice between, on the one hand, a tracking service during real surgery as per the foregoing embodiments and per co-pending patent applications PCT/IL2012/000363 and U.S. patent application Ser. No. 13/571,284, and, on the other hand, a haptic feedback service for virtual surgery based on the model derived from the same patient scan data using the haptic device for physical sensory haptic feedback. This user selectable choice allows the user the option of first planning the surgery and executing it using haptic feedback technology in virtual space on a model of the same surgical site, before committing to the real surgery in real space on the real surgical site.

In one embodiment the virtual surgery may be undertaken removed from the real surgical site, but nevertheless on the same model based on the same scan data of the surgical site as what is to be used in the real surgery. In this specification the term “ex situ virtual surgery” is used to describe this arrangement.

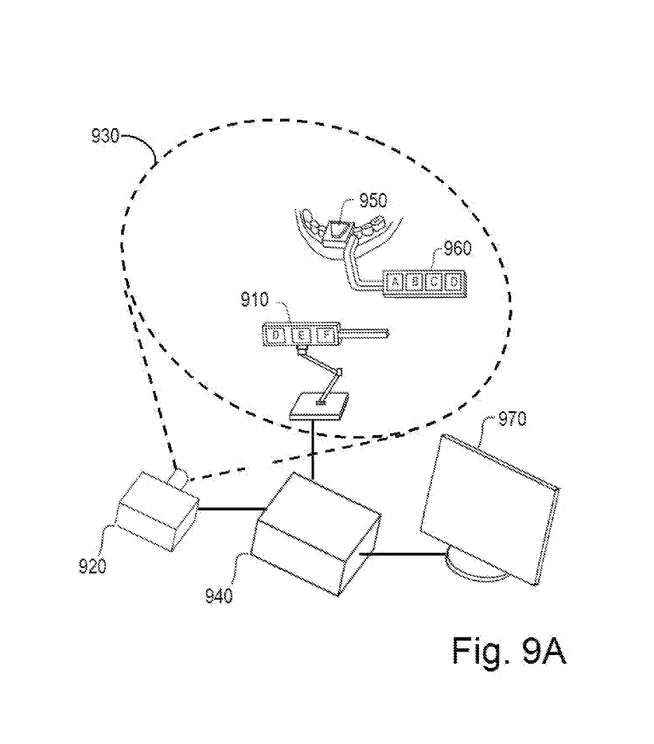

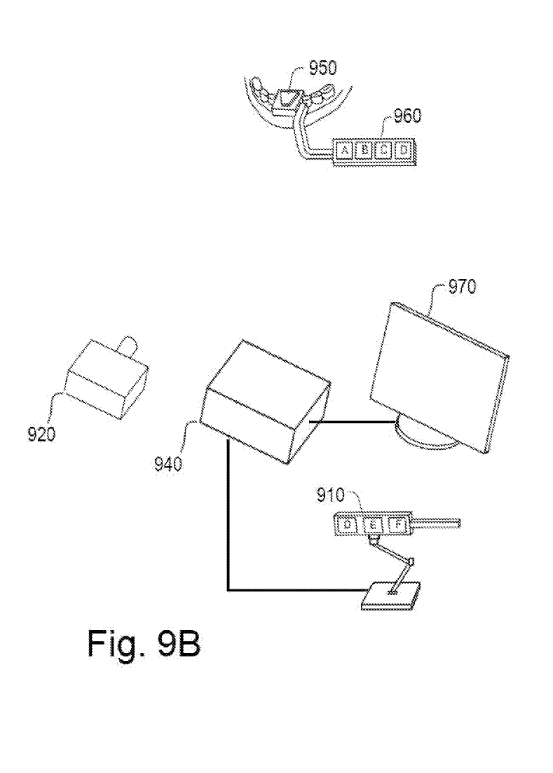

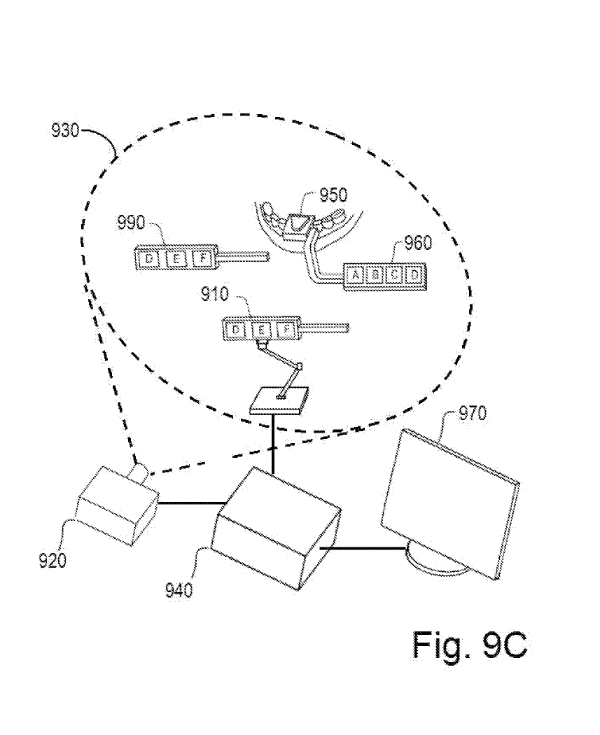

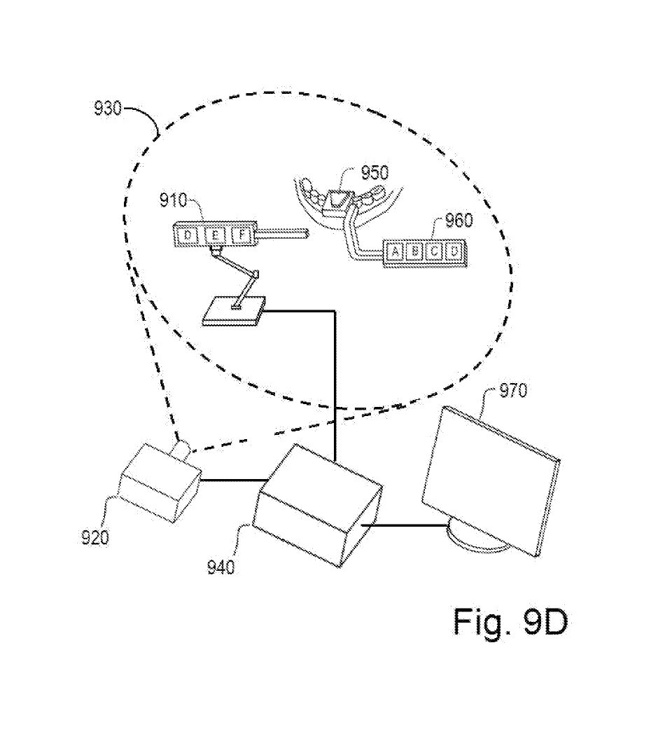

In another embodiment shown in FIG. 9A, the virtual surgery may be undertaken at the actual surgical site, but with the haptic device substituting for the actual surgical instrument. In this specification the term “in situ virtual surgery” is used to describe this latter arrangement. In this in situ virtual surgery implementation, any one of the tracking systems described in the various foregoing embodiments as per co-pending patent applications PCT/IL2012/000363 and U.S. patent application Ser. No. 13/571,284 may be employed so that the virtual surgery is undertaken under more realistic conditions in which the surgical site is subject to change during surgery. To facilitate this particular embodiment, haptic device 910 is provided with a suitable tracking marker that may be tracked by tracker 920 of the system described in FIGS. 3A-J, FIG. 5 and FIG. 6. Considering now FIG. 9A, haptic device 910 is operated within the field of view 930 of tracker 920. The surgical site, chosen to be a dental surgery site in the present example, is within the same field of view 930. Controller 940 keeps track of the variation in position and orientation of fiducial 950 by means of tracking marker 960 and the varying model of the surgical site is displayed in real time on monitor 970.

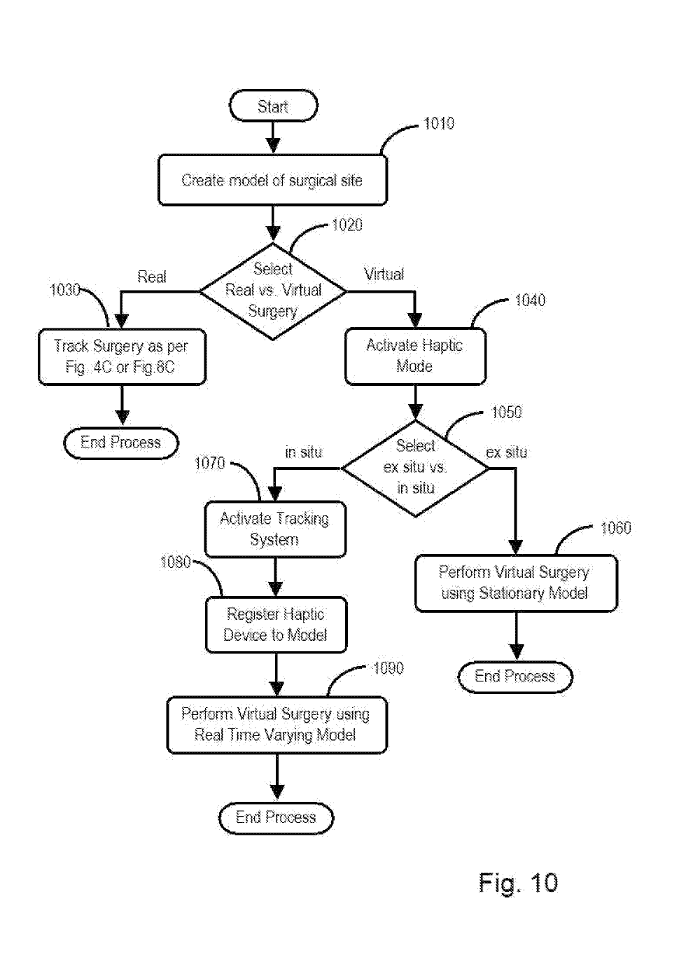

A method of use of the system is described at the hand of FIG. 10. In respect of the creation of the model of the surgical site from the scan, the method proceeds similarly as for the virtual surgery as for the real surgery, as described in FIGS. 4A an 4B for a single element fiducial reference of FIGS. 3A-J, 5 and 6, as well as FIGS. 8A and 8B for a multi-element fiducial as per FIGS. 7A and 7B. In FIG. 10 these collections of actions from FIGS. 4A and 4B, or 8A and 8B, are collectively represented by the creation of the model of the surgical site [at 1010]. Once the model of the surgical site is in existence, the method proceeds as follows.

The user selects [at 1020] between real surgery and virtual surgery. If real surgery is selected, then the method proceeds by tracking the surgery [at 1030] as per FIGS. 4C or 8C, depending on whether the single element fiducial of FIGS. 3A-J, 5 and 6 was selected or the multi-element fiducial of FIGS. 7A and 7B, respectively.

If virtual surgery is selected at [1020] then the method proceeds with controller 940 of FIGS. 9A and 9B, for example processor 214 and memory 217 of computer 210 of FIG. 2, activating [at 1040] the haptic mode. The user selects [at 1050] between ex situ virtual surgery and in situ virtual surgery.

If ex situ virtual surgery is selected at [1050], haptic device 910 may be used as user interface or “hand piece” to perform the ex situ virtual surgery [at 1060]. In the case of ex situ virtual surgery, shown in FIG. 9B, there is no requirement for tracking the “hand piece” as there is no direct relationship between haptic device 910 and any images captured by tracker 920. The virtual surgery is performed using a stationary model of the surgical site, as the data from the real site is not being updated. This is represented in FIG. 9B by tracker 920 not having a field of view, since it may optionally be turned off. There is also no need in this particular mode for a data connection between controller 940 and tracker 920.

If in situ virtual surgery is selected [at 1050], as represented by FIG. 9A, then controller 940 activates [at 1070] the tracking system comprising tracker 920 and tracking markers, such as tracking marker 960 and haptic device 910. In particular, haptic device 910, which is positioned proximate the surgery site for this form of virtual surgery, may be tracked in real time by tracker 920. This can be done either using a tracking marker attached to haptic device 910, or using the digital position and orientation data generated by haptic device 910. To this end the position and orientation of haptic device 910 is registered with respect to the real time model of the surgery site [at 1080] via image information gathered by tracker 920. A tracking marker attached to haptic device 910 may be used for this purpose, since the tracking markers of the system are specifically designed for position and orientation detection. The virtual working tip of the virtual instrument represented by haptic device 910, for example a virtual dental drill, may be positioned in software to be at the same position as the working tip of the corresponding real instrument (the dental drill in the dental surgery example). With the virtual instrument registered, the in situ virtual surgery may be performed [at 1090] while the monitoring system displays the position and orientation of the virtual instrument in real time on display monitor 970, for example display 224 of FIG. 2.

Haptic device 910 and a corresponding actual surgical device 990 may be in field of view 930 of tracker 920 at the same time, as shown in FIG. 9C. This allows rapid switching between real surgery and virtual surgery.

The grip of haptic device 910 may be of the same or similar shape as that of the real instrument that is to be used in the corresponding real surgery and, as shown in FIG. 9D, haptic device 910 may be positioned with respect to the surgical site exactly where the grip of surgical instrument 990 is to be manipulated by the user during real surgery. The benefit of this approach is that real surgery may be interrupted by switching to in situ virtual surgery to practice a further surgical step in virtual space, before switching back to the monitoring or tracking mode to undertake the actual surgery using a tracked surgical instrument as per the foregoing embodiments. There is no limitation on the placement of haptic device 910 within the field of view of the tracker beyond the constraints of safety and the visibility of the haptic and its attached tracking marker device to the tracker. Therefore, placing haptic device 910 in the same position as the real instrument is merely one particular choice of “in situ” location, though a very significant one in that it adds to the realism of the virtual surgery for the user, who may be the relevant surgeon. In general, haptic device 910 may be placed anywhere in the field of view of tracker 920, thereby allowing controller 940 to provide real time monitoring of the virtual surgical instrument within a real time varying representation of the surgical site on the display.

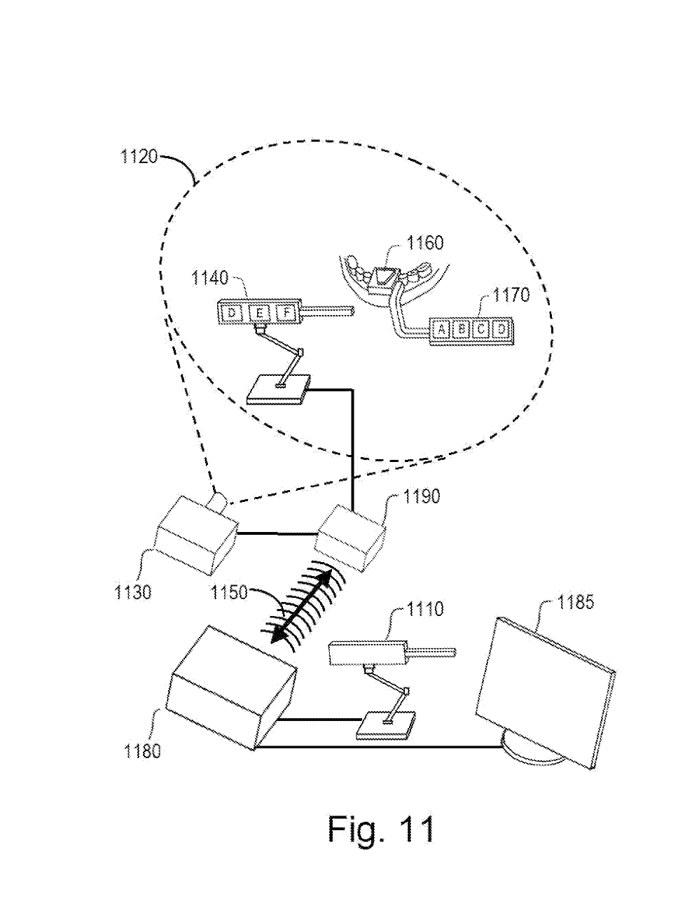

In a further embodiment, described relating to FIG. 11, haptic device 1110 may be located at a site remote from the surgical site, and thereby clearly outside field of view 1120 of tracker 1130. This arrangement, described in the present specification by the term “remote surgery,” refers to when the user or surgeon may be in a different geographic location than the surgical site. Signals from haptic device 1110 may be transmitted to remotely manipulable surgical instrument 1140 located proximate the surgical site via communications link 1150. Communications link 1150 may be any one or more of wired, optical, radio, microwave or satellite, or any other communications link of suitable bandwidth, and it is arranged to transmit data between the remote location of haptic device 1110 and the elements of the system located proximate the surgical site. Fiducial reference 1160 and its attached tracking marker 1170 also are disposed within field of view 1120 of tracker 1130. Signals between remotely manipulable surgical instrument 1140 and controller 1180, as well as signals between tracker 1130 and controller 1180, may be collected, distributed, received and transmitted on communications link 1150 by a suitable data hub 1190. Controller 1180 may display information or imagery related to the model of the surgical site, and/or imagery of the surgical site itself, on monitor 1185.

Remotely manipulable surgical instrument 1140 may be electronically slaved to haptic device 1110 via controller 1180 and communications link 1150, thereby to allow surgical instrument 1140 to reproduce every motion made by haptic device 1110. In this embodiment the user, who may be a surgeon, may conduct the surgery from the remote location while observing the progress of the surgery on display monitor 1185 in his proximity. Remotely manipulable surgical instrument 1140 may be fitted with a tracking marker to allow it to be tracked by tracker 1130 proximate the surgical site. This allows remotely manipulable surgical instrument 1140 to be tracked in real time with respect to the real surgical site even as the surgical site varies. As in the other embodiments, the model may be displayed in real time to the user on monitor 1185 at the user's remote location and the user may follow the relative motion of remotely manipulable surgical instrument 1140 as controlled by him using haptic device 1110 located in his proximity.

This embodiment may be arranged so that the user, who may be a surgeon, may elect to do virtual surgery. In this respect, the system may be arranged to make the mode of surgery, real or virtual, selectable. If virtual surgery is selected, the user may stop the motion of remotely manipulable surgical instrument 1140 to allow him- or herself the opportunity to undertake virtual surgery at his/her own location using haptic device 1110 and the model based on the scan data. Since haptic device 1110 is still registered to remotely manipulable surgical instrument 1140 at the time of haptic device 1140 being stopped, any subsequent motion or reorientation of haptic device 1110 may be expressed within the system as a relative position and orientation with respect to stationary remotely manipulable surgical instrument 1140. Since the location and orientation of stationary surgical instrument 1140 is known to controller 1180, based on its tracking marker, the position and orientation of haptic device 1110 is known with respect to the model of the surgical site. With the real time information from tracker 1130 still being supplied to controller 1180, the user may still conduct virtual surgery within the model, with the model giving a real time representation of the surgical site as it varies over time.

The user may optionally base the virtual surgery on the stationary model of the surgical site, as derived from the original scan of the surgical site. In this process he or she forgoes the benefit of any real time variation of the surgical site within the virtual surgery. The system may be arranged to allow the choice between real time and stationary virtual surgery to be selectable to the user. The three method options within the overall remote surgery arrangement may be wholly selectable on the part of the user and may be implemented in one embodiment of the surgical monitoring system and method.

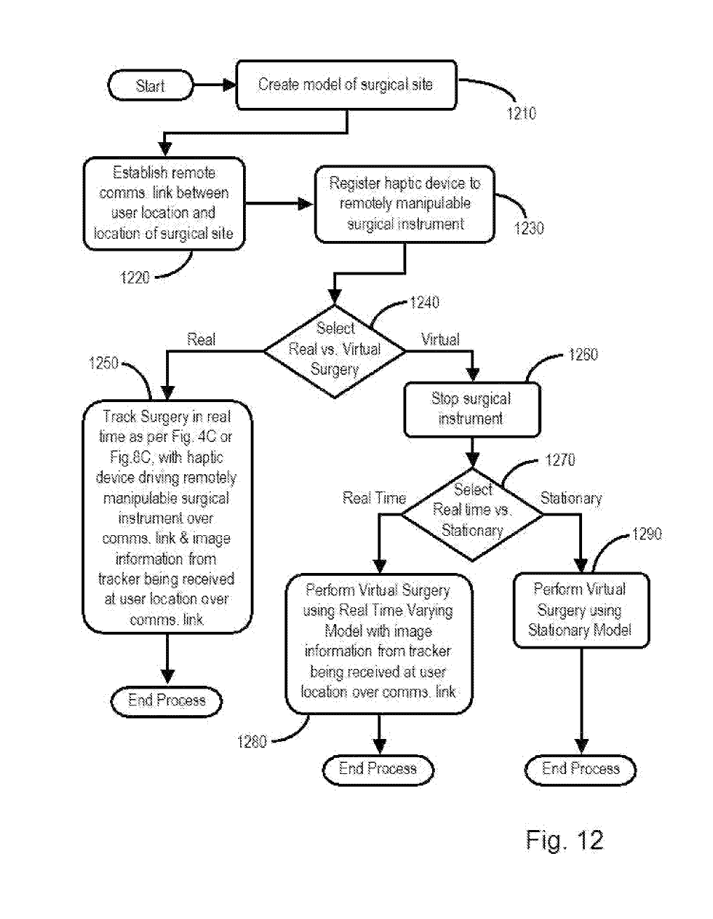

The “remote surgery” method embodiment may be described as follows in terms of the flow diagram of FIG. 12, based on the apparatus of FIG. 11. The model of the surgical site is created [at 1210] in the same way as with the embodiments of FIGS. 4A and 4B and FIGS. 8A and 8B. Communications link 1150 is established [at 1220] between the location of the user and the location of the surgical site. This link is used to communicate image information about the surgical site and the model derived therefrom to the location of the user, and is also used to communicate information from haptic device 1110 to the surgical site, where it is to be used to guide remotely manipulable surgical device 1140. Haptic device 1140 is registered [at 1230] to remotely manipulable surgical device 1140 and thereby to the surgical site.

The user may select [at 1240] whether real or virtual surgery is to be undertaken. If the surgery is selected to be real [at 1240], then the surgery is tracked [at 1250] by tracker 1130 in real time as per FIG. 4C or FIG. 8C, with haptic device 1110 driving remotely manipulable surgical instrument 1140 over communications link 1150 between controller 1180 and hub 1190, while image information from tracker 1130 is sent in the opposite direction over communications link 1150 to controller 1180. As the surgery proceeds, the model is constantly updated based on tracking markers (for example 1170) proximate the surgical site, including any tracking marker on the surgical instrument, and thereby allows the model of the surgical site to be adapted in real time on monitor 1185 at the location of the user, who may be a surgeon.

If the surgery is selected to be virtual [at 1240], then surgical instrument 1140 proximate the surgical site is stopped [at 1260] and the user may select [at 1270] between real time virtual surgery and stationary virtual surgery. If real time virtual surgery is selected [at 1270] then the system may perform [at 1280] virtual surgery using the real time varying model while image information from tracker 1130 is received at the user location over communications link 1150. If stationary virtual surgery is selected [at 1270] then the system may perform [at 1290] virtual surgery using the stationary model as no image information from tracker 1130 is used to update the model in real time.

In another aspect of the invention, all of the apparatus and method described relating to FIGS. 9A, 9B, 9C, 9D, 10, 11 and 12 may be implemented with haptic device 910, 1110 replaced by a digital manipulator device without haptic feedback, or the haptic feedback facility of haptic device 910, 1110 may simply be turned off or disengaged. In the latter case the use of haptic feedback may therefore be an operational option. A suitable digital manipulator device may have six degrees of freedom in three dimensions, three being three translational and three rotational motions. Selecting haptic mode [at 1040] in FIG. 10 is obviated in the absence of the manipulator having haptic feedback.

While this invention has been described as having an exemplary design, the present invention may be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains.

What is claimed is:

1. A system for monitoring a surgical site and a surgical instrument, comprising:
a tracker adapted to monitor the surgical site, the tracker having a field of view;
a fiducial reference attached proximate the surgical site;
a first tracking marker attached to the fiducial reference in a predetermined orientation, the first tracking marker disposed within the field of view;
a digital manipulator device configured for providing three-dimensional location and orientation information relating to the position of said digital manipulator device; and
a controller having a processor and memory, said memory storing a plurality of instructions, said plurality of instructions when executed by said processor to create a model of the surgical site from scan data of the surgical site; selectively update the model of the surgical site based on image information from the tracker about the surgical site; obtain the three-dimensional location and orientation information of the digital manipulator device; and display simultaneously on a display monitor the model of the surgical site and one of a virtual representation of the digital manipulator device and a virtual representation of the surgical instrument.

2. The system of claim 1 wherein the digital manipulator is configured to provide the three-dimensional location and orientation information in real time and the controller is configured to obtain and display in real time.

3. The system of claim 1 wherein the fiducial reference is structured and arranged to move with the surgical site.

4. The system of claim 3 wherein the fiducial reference is structured and arranged to attach to tissue proximate the surgical site.

5. The system of claim 1 further comprising a second tracking marker attached to the surgical instrument for registering the surgical instrument to the surgical site.

6. The system of claim 2 wherein the digital manipulator device bears a third tracking marker disposed within the field of view for registering the digital manipulator device to the surgical site; and
the controller is further configured for obtaining real time three-dimensional location and orientation information of the digital manipulator device from the real time image information.

7. The system of claim 1 wherein the digital manipulator device is a haptic device and the controller is configured for allowing the user to optionally select haptic feedback, the haptic feedback based on the model.

8. The system of claim 1 further comprising a remote communications link, and wherein the controller and the digital manipulator device are disposed remote from the surgical site, the surgical instrument is a remotely operable surgical instrument; and the remote communications link is configured for
transmitting image information from the tracker to the controller,
transmitting three-dimensional location and orientation information about the remotely operable surgical instrument to the controller; and for
transmitting control information from the controller to the remotely operable surgical instrument.

9. The system of claim 8 wherein the remotely operable surgical instrument bears a fourth tracking marker disposed within the field of view for registering the remotely operable surgical instrument to the surgical site; and the controller is further configured for obtaining three-dimensional location and orientation information of the remotely operable surgical instrument from the image information.

10. A method for monitoring a surgery at a surgical site using a surgical instrument, the surgical instrument bearing a first tracking marker disposed within a field of view of a tracker, the tracker disposed proximate the surgical site, the method comprising,
affixing a fiducial reference proximate the surgical site;
obtaining scan data of the surgical site;
creating a model of the surgical site from the scan data;
attaching a second tracking marker to the fiducial reference within the field of view;
registering a digital manipulator device to the model;
obtaining the three-dimensional location and orientation information of the digital manipulator device; and
displaying simultaneously on a display monitor the model of the surgical site and one of a virtual representation of the digital manipulator device and a virtual representation of the surgical instrument.

11. The method of claim 10 wherein the affixing step involves having the fiducial reference affixed in a manner allowing the fiducial reference to move in conjunction with the surgical site.

12. The method of claim 11 wherein the affixing step involves having the fiducial reference affixed to tissue proximate the surgical site.

13. The method of claim 10 further including the step of updating in real time the model based on image information about the surgical site from the tracker.

14. The method of claim 10 wherein the steps of obtaining and displaying are done in real time.

15. The method of claim 10 wherein the step of registering the digital manipulator device to the model includes registering to the model a third tracking marker attached to the digital manipulator device, the third tracking marker being disposed within the field of view.

16. The method of claim 10 wherein the real time three-dimensional location and orientation information of the digital manipulator device is obtained from the image information.

17. The method of claim 10 further comprising the step of controlling the position and orientation of the surgical instrument over a remote communications link by manipulation of the digital manipulator device, wherein the surgical instrument is a remotely operable surgical instrument configured for receiving control instructions from a controller based on position and orientation information from the digital manipulator device.

18. The method of claim 17 further comprising sending the image information from the tracker to the controller over the remote communications link.

19. The method of claim 10 further comprising the step of providing haptic feedback to a user via the digital manipulator device based on the model.

20. The method of claim 19 wherein the step of providing haptic feedback may be selectively activated.
